(19) United States
(12) Patent Application Publication
(10) Pub. No.: US 2026/0217847 A1
(43) Pub. Date: Jul. 30, 2026

(54) ANTI-TFR1 SINGLE-DOMAIN ANTIBODY AND USE THEREOF

(71) Applicant: NANJING REGENECORE BIOTECH CO., LTD., Nanjing (CN)

(72) Inventors: Zhipeng SU, Nanjing (CN); Yang WANG, Nanjing (CN); Wei XIE, Nanjing (CN)

(73) Assignee: NANJING REGENECORE BIOTECH CO., LTD., Nanjing (CN)

(21) Appl. No.: 19/145,904

(22) PCT Filed: Feb. 17, 2023
(86) PCT No.: PCT/CN2023/076734

§ 371 Date: Jul. 4, 2025

(51) Int. Cl. C07K 16/28; A61K 39/00; A61K 39/385; A61K 47/68; C07K 14/705; G01N 33/575

(52) U.S. Cl. CPC C07K 16/2881; A61K 39/385; A61K 47/6849; C07K 14/70582; G01N 33/5759; A61K 2039/6031; C07K 2317/14; C07K 2317/24; C07K 2317/565; C07K 2317/567; C07K 2317/569; C07K 2317/77; G01N 2333/70582

(57) ABSTRACT

The present invention provides an anti-TfR1 single-domain antibody and a use thereof. The single-domain antibody includes a heavy chain variable region, wherein the heavy chain variable region includes a heavy chain CDR1 as shown in any one of SEQ ID NO: 53-SEQ ID NO: 60, a heavy chain CDR2 as shown in any one of SEQ ID NO: 61-SEQ ID NO: 72, and a heavy chain CDR3 as shown in any one of SEQ ID NO: 73-SEQ ID NO: 82.

### CROSS-REFERENCE TO RELATED APPLICATIONS

This application is an U.S. national phase application under 35U.S.C. § 371 based upon international patent application No. PCT/CN2023/076734 filed on Feb. 17, 2023. The content of which is hereby incorporated by reference in its entirety.

### SEQUENCE LISTING

The instant application contains a Sequence Listing which has been submitted in XML format via EFS-Web and is hereby incorporated by reference in its entirety. Said XML copy is named LYHJ0109_Sequence_Listing.xml, created on Jul. 4, 2025, and is 96,106 bytes in size.

### TECHNICAL FIELD

The present disclosure relates to the field of biomedicine. Specifically, the present disclosure relates to transferrin receptor 1 (TfR1) antigen epitope peptides, TfR1 antigens, anti-TfR1 single-domain antibodies, recombinant proteins, antibody complexes, pharmaceutical compositions, kits, and uses thereof for preventing or treating diseases.

### BACKGROUND

Transferrin receptor (TfR) is a type II homodimeric transmembrane glycoprotein consisting of two identical 90 kDa subunits linked by two disulfide bridges (Jing and Trowbridge 1987, McClelland et al., 1984). Each TfR molecule has a short cytoplasmic N-terminal domain of 61 amino acids containing a YTRF (Tyrosine-Threonine-Arginine-Phenylalanine) internalization motif, a single hydrophobic transmembrane segment of 27 amino acids, and a large C-terminal extracellular domain of 670 amino acids, containing a trypsin cleavage site and a transferrin binding site (Aisen, 2004). Each subunit of TfR molecule is capable of binding a transferrin molecule. The extracellular domain of TfR molecule has one O-glycosylation site and three N-glycosylation sites, the latter being of great importance for the proper folding and transport of TfR molecule to the cell surface (Hayes et al., 1997). There are also palmitylation sites in the intramembranous domain of TfR molecule. It is presumed that these palmitoylation sites enable TfR to be anchored and internalized (Alvarez et al., 1990, Omary and Trowbridge, 1981). Many studies have shown that the expression level of TfR is higher in cancer cells compared to in healthy cells and is correlated with tumor grade and disease stage or prognosis.

There are two types of transferrin receptors, including the TfR1 receptor and a homologous receptor, TfR2, expressed primarily in the liver. It has been reported that the expression level of TfR1 receptor in malignant proliferative cells is significantly higher than that in normal cells. TfR1 is considered to be an important target for tumor treatment. In recent years, TfR1 receptor is also considered to be an important target for drug transport across the blood-brain barrier.

However, there is still room for improvement in antibodies that specifically recognize transferrin receptor TfR1.

### SUMMARY

A first aspect of the present disclosure provides a human TfR1 antigen epitope peptide, wherein the TfR1 antigen epitope peptide has an amino acid sequence selected from: (1) SEQ ID NO: 85; (2) SEQ ID NO: 97; (3) SEQ ID NO: 98; (4) SEQ ID NO: 100; and (5) SEQ ID NO: 101. The TfR1 antigen epitope peptide can be used to prepare an anti-TfR1 antibody that specifically binds to the TfR1 protein with high affinity.

A second aspect of the present invention provides a TfR1 antigen, which is a conjugate of the human TfR1 antigen epitope peptide as described in the first aspect to a carrier protein. The TfR1 antigen can be used to prepare an anti-TfR1 antibody that specifically binds to the TfR1 protein with high affinity.

A third aspect of the present invention provides a human TfR1 antibody, which is an anti-TfR1 antibody produced using the TfR1 antigen as described in the second aspect as an immunogen.

A fourth aspect of the present invention provides an anti-TfR1 single-domain antibody, the anti-TfR1 single-domain antibody including a heavy chain variable region, wherein the heavy chain variable region includes a heavy chain complementarity determining region 1 (CDR1) as set forth in any one of SEQ ID NO: 53 to SEQ ID NO: 60, a heavy chain complementarity determining region 2 (CDR2) as set forth in any one of SEQ ID NO: 61 to SEQ ID NO: 72, and a heavy chain complementarity determining region 3 (CDR3) as set forth in any one of SEQ ID NO: 73 to SEQ ID NO: 82. The anti-TfR1 single-domain antibody can specifically bind to the TfR1 protein with high affinity.

In some embodiments, the heavy chain CDR1, the heavy chain CDR2, and the heavy chain CDR3 have amino acid sequences selected from the group consisting of (1) to (12):
(1) CDR1 as set forth in SEQ ID NO:53, CDR2 as set forth in SEQ ID NO:62, and CDR3 as set forth in SEQ ID NO:76;
(2) CDR1 as set forth in SEQ ID NO:54, CDR2 as set forth in SEQ ID NO:72, and CDR3 as set forth in SEQ ID NO:80;
(3) CDR1 as set forth in SEQ ID NO:55, CDR2 as set forth in SEQ ID NO:64, and CDR3 as set forth in SEQ ID NO:73;
(4) CDR1 as set forth in SEQ ID NO:56, CDR2 as set forth in SEQ ID NO:61, and CDR3 as set forth in SEQ ID NO:75;
(5) CDR1 as set forth in SEQ ID NO: 57, CDR2 as set forth in SEQ ID NO: 66, and CDR3 as set forth in SEQ ID NO: 74;
(6) CDR1 as set forth in SEQ ID NO:58, CDR2 as set forth in SEQ ID NO:68, and CDR3 as set forth in SEQ ID NO:78;
(7) CDR1 as set forth in SEQ ID NO:59, CDR2 as set forth in SEQ ID NO:67, and CDR3 as set forth in SEQ ID NO:82;
(8) CDR1 as set forth in SEQ ID NO:59, CDR2 as set forth in SEQ ID NO:63, and CDR3 as set forth in SEQ ID NO:81;
(9) CDR1 as set forth in SEQ ID NO:59, CDR2 as set forth in SEQ ID NO:71, and CDR3 as set forth in SEQ ID NO:77;
(10) CDR1 as set forth in SEQ ID NO:59, CDR2 as set forth in SEQ ID NO:69, and CDR3 as set forth in SEQ ID NO:79;
(11) CDR1 as set forth in SEQ ID NO:59, CDR2 as set forth in SEQ ID NO:70, and CDR3 as set forth in SEQ ID NO:77; and
(12) CDR1 as set forth in SEQ ID NO:60, CDR2 as set forth in SEQ ID NO:65, and CDR3 as set forth in SEQ ID NO:73.

In some embodiments, the heavy chain variable region further includes framework regions (FRs). The framework regions (FRs) include FR1, FR2, FR3 and FR4 regions.

In some embodiments, the FR1 region has an amino acid sequence as set forth in any one of SEQ ID NOS: 25-33 or having at least about 60%, about 65%, about 70%, about 75%, about 80%, about 85%, about 90%, about 95% sequence homology to any one of SEQ ID NOS: 25-33. Optionally, the amino acid sequence of the FR1 region includes up to 5 amino acid substitutions relative to any one of SEQ ID NOS: 25-33.

In some embodiments, the FR2 region has an amino acid sequence as set forth in any one of SEQ ID NOS: 34-40 or having at least about 60%, about 65%, about 70%, about 75%, about 80%, about 85%, about 90%, about 95% sequence homology to any one of SEQ ID NOS: 34-40. Optionally, the amino acid sequence of the FR2 region includes up to 5 amino acid substitutions relative to any one of SEQ ID NOS: 34-40.

In some embodiments, the FR3 region has an amino acid sequence as set forth in any one of SEQ ID NOS: 41-51 or having at least about 60%, about 70%, about 72%, about 74%, about 76%, about 78%, about 80%, about 82%, about 85%, about 88%, about 90%, about 92%, about 94%, about 96% sequence homology to any one of SEQ ID NOS: 41-51. Optionally, the amino acid sequence of the FR3 region includes up to 10 amino acid substitutions relative to any one of SEQ ID NOS: 41-51.

In some embodiments, the FR4 region has an amino acid sequence as set forth in SEQ ID NO: 52 or having at least about 60%, about 65%, about 70%, about 75%, about 80%, about 85%, about 90% sequence homology to SEQ ID NO: 52. Optionally, the amino acid sequence of the FR4 region includes up to 5 amino acid substitutions relative to SEQ ID NO: 52.

The present inventors have obtained a variety of anti-TfR1 single-domain antibodies through extensive research. These anti-TfR1 single-domain antibodies may have a wide range of affinities for TfR1 protein and thus have extensive applications.

In some embodiments, the anti-TfR1 single-domain antibody has an amino acid sequence as set forth in any one of SEQ ID NOS: 1-12, or having at least about 80% sequence homology to any one of SEQ ID NOS: 1-12. The anti-TfR1 single-domain antibody can specifically bind to the TfR1 protein with high affinity.

In some embodiments, the amino acid sequence of the anti-TfR1 single-domain antibody is as set forth in any one of SEQ ID NOS: 1-12. Alternatively, the amino acid sequence of the anti-TfR1 single-domain antibody has at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% sequence homology to any one of SEQ ID NOS: 1 to 12, and the anti-TfR1 single-domain antibody is capable of specifically binding to the TfR1 protein.

In some embodiments, the anti-TfR1 single-domain antibody of the present disclosure may be an Fc fusion antibody, a humanized antibody, a chimeric antibody, a monoclonal antibody, or a polyclonal antibody.

A fifth aspect of the present disclosure provides a recombinant protein including the amino acid sequence of the anti-TfR1 single-domain antibody as described in the fourth aspect.

In some embodiments, the recombinant protein can be a multi-epitope antibody, a multivalent antibody, or a multispecific antibody.

A sixth aspect of the present disclosure provides an antibody complex, including the anti-TfR1 single-domain antibody as described in the fourth aspect or the recombinant protein as described in the fifth aspect.

In some embodiments, the antibody complex can be a conjugate formed by conjugating the anti-TfR1 single-domain antibody as described in the fourth aspect or the recombinant protein as described in the fifth aspect to an active ingredient selected from a therapeutic agent, a diagnostic agent, a carrier protein and a liposome.

A seventh aspect of the present disclosure provides a nucleic acid encoding the anti-TfR1 single-domain antibody as described in the fourth aspect or the recombinant protein as described in the fifth aspect.

In some embodiments, the nucleic acid has a nucleotide sequence as set forth in SEQ ID NOS: 13-24. Alternatively, the nucleotide sequence of the nucleic acid has at least about 80% sequence homology to any one of SEQ ID NO: 13-24, such as at least about 85% homology, at least about 90% homology, at least about 95% homology, at least about 98% homology or at least about 99% homology, and the anti-TfR1 single-domain antibody encoded by the nucleic acid can specifically bind to the TfR1 protein.

An eighth aspect of the present disclosure provides an expression vector including the nucleic acid as described in the seventh aspect. The expression vector expresses the anti-TfR1 single-domain antibody as described in the fourth aspect or the recombinant protein as described in the fifth aspect.

A ninth aspect of the present disclosure provides a host cell, including the expression vector as described in the eighth aspect. The host cell expresses the anti-TfR1 single-domain antibody as described in the fourth aspect or the recombinant protein as described in the fifth aspect.

A tenth aspect of the present disclosure provides a pharmaceutical composition, including the anti-TfR1 single-domain antibody as described in the fourth aspect, the recombinant protein as described in the fifth aspect, the antibody complex as described in the sixth aspect, the nucleic acid as described in the seventh aspect, the expression vector as described in the eighth aspect, or the host cell as described in the ninth aspect, and a pharmaceutically acceptable carrier. The pharmaceutical composition is for use in preventing or treating a disease.

An eleventh aspect of the present disclosure provides use of the anti-TfR1 single-domain antibody as described in the fourth aspect, the recombinant protein as described in the fifth aspect, the antibody complex as described in the sixth aspect, the nucleic acid as described in the seventh aspect, the expression vector as described in the eighth aspect, the host cell as described in the ninth aspect, or the pharmaceutical composition as described in the tenth aspect in manufacturing a medicament for preventing or treating a disease associated with abnormal TfR1 receptor expression.

A twelfth aspect of the present disclosure provides an anti-TfR1 single-domain antibody as described in the fourth aspect, a recombinant protein as described in the fifth aspect, an antibody complex as described in the sixth aspect, a nucleic acid as described in the seventh aspect, an expression vector as described in the eighth aspect, a host cell as described in the ninth aspect, or a pharmaceutical composition as described in the tenth aspect for use as a medicament.

In this aspect, the present disclosure further provides an anti-TfR1 single-domain antibody as described in the fourth aspect, a recombinant protein as described in the fifth aspect, an antibody complex as described in the sixth aspect, a nucleic acid as described in the seventh aspect, an expression vector as described in the eighth aspect, a host cell as described in the ninth aspect, or a pharmaceutical composition as described in the tenth aspect for use in preventing or treating a disease associated with abnormal TfR1 receptor expression.

A thirteenth aspect of the present disclosure provides a method for preventing or treating a disease associated with abnormal TfR1 receptor expression, including administering to a subject in need thereof a therapeutically effective amount of the anti-TfR1 single-domain antibody as described in the fourth aspect, the recombinant protein as described in the fifth aspect, the antibody complex as described in the sixth aspect, the nucleic acid as described in the seventh aspect, the expression vector as described in the eighth aspect, the host cell as described in the ninth aspect, or the pharmaceutical composition as described in the tenth aspect.

In some embodiments, the disease associated with abnormal TfR1 receptor expression is a tumor that abnormally expresses the TfR1 receptor.

In some embodiments, the tumor includes but is not limited to breast cancer, glioma, lung adenocarcinoma, leukemia, lymphoma, non-Hodgkin's lymphoma, ovarian cancer, or the like.

A fourteenth aspect of the present disclosure provides a kit for detecting the TfR1 receptor, including the anti-TfR1 single-domain antibody as described in the fourth aspect, the recombinant protein as described in the fifth aspect, the antibody complex as described in the sixth aspect, the nucleic acid as described in the seventh aspect, the expression vector as described in the eighth aspect, the host cell as described in the ninth aspect, or the pharmaceutical composition as described in the tenth aspect.

In some embodiments, the kit is for use in diagnosing a disease associated with abnormal TfR1 receptor expression.

A fifteenth aspect of the present disclosure provides the anti-TfR1 single-domain antibody as described in the fourth aspect, the recombinant protein as described in the fifth aspect, or the antibody complex as described in the sixth aspect for use as a drug for crossing blood-brain barrier.

It should be understood that the above-mentioned technical features of the present disclosure and the technical features described below (such as embodiments) can be combined with each other within the scope of the present disclosure, thereby forming new technical solutions. Due to space limitations, they will not be elaborated here.

### BRIEF DESCRIPTION OF THE DRAWINGS

In order to more clearly illustrate the technical solution of the present invention, the drawings required for use in the embodiments are briefly described below. Obviously, for those of ordinary skill in the art, other drawings can be obtained based on these drawings without paying any creative work.

FIG. 1 is an SDS-PAGE electrophoretogram of the purified humanized TfR1 proteins in Example 1 of the present disclosure.

FIG. 2 is a diagram showing library enrichment for screening of antibodies targeting humanized TfR1 protein in Example 3 of the present disclosure.

FIG. 3 is a dose-effect curve and measurement results of antibody-antigen binding in Example 12 of the present disclosure.

FIG. 4 is a dose-effect curve and measurement results of antibody-antigen binding in Example 12 of the present disclosure.

FIG. 5 is a dose-effect curve and measurement results of antibody-antigen binding in Example 12 of the present disclosure.

FIG. 6 is a dose-effect curve and measurement results of antibody-antigen binding in Example 12 of the present disclosure.

FIG. 7 is a dose-effect curve and measurement results of antibody-antigen binding in Example 12 of the present disclosure.

FIG. 8 is a dose-effect curve and measurement results of antibody-antigen binding in Example 12 of the present disclosure.

### DETAILED DESCRIPTION

In the present disclosure, the technical solutions in the embodiments of the present disclosure will be clearly and completely described below in conjunction with the drawings in the embodiments of the present disclosure. Obviously, the described embodiments are only part of the embodiments of the present disclosure, rather than all the embodiments. Based on the embodiments in the present disclosure, all other embodiments obtained by those of ordinary skill in the art without creative work fall within the scope of protection of the present disclosure.

### Definitions

As used herein, “single-domain antibody” (sdAb, also known as nanobody or VHH) refers to an antibody that includes only the heavy chain variable region of a heavy chain antibody. It is the smallest functional antigen-binding fragment. A single-domain antibody can be a natural antibody with only a heavy chain variable domain, a single-domain antibody derived from a conventional antibody, and an engineered single-domain antibody.

As used herein, the term “Fc fusion antibody” refers to a novel protein produced by fusing the Fc segment of an antibody of interest with a functional protein molecule having biological activity using genetic engineering technology.

A “humanized antibody” refers to an antibody obtained by fusing the heavy chain variable domain of an antibody of interest (such as an animal antibody) with the constant region of a human antibody, an antibody obtained by grafting the complementarity-determining region (sequences of CDR1, CDR2, and CDR3) of an antibody of interest into the variable region of a human antibody, or an antibody obtained by subjecting an antibody of interest to individual amino acid mutations according to the structures of the framework region (FR1, FR2, FR3, and FR4) of a human antibody. The humanized antibody may be obtained through synthesis or site-directed mutagenesis.

The term “multi-epitope antibody” refers to an antibody that has the ability to specifically bind to two or more different epitopes on the same or different targets.

The term “multivalent antibody” refers to an antibody that can bind to multiple antigenic epitopes. The number of antigenic epitopes that the antibody binds to is called the antigen binding valency.

The term “multispecific antibody” refers to an artificial antibody containing two or more specific antigen-binding sites. The antibody can stimulate a targeted immune response in a host (e.g., human).

Herein, the terms “TfR1” is used interchangeably with “TfR1 protein”, “TfR1 receptor” or “TfR1 antigen”, all of which refer to transferrin receptor 1, which can be specifically bound by the anti-TfR1 single-domain antibody of the present disclosure.

The term “tumor” refers to a group of diseases, which can be defined as any new growth of abnormal benign or malignant tissue that possesses no physiological function and arises from usually rapid uncontrolled cellular proliferation and has the potential to invade or spread to other parts of the body.

The term “subject” is used interchangeably with “patient” and usually refers to humans, but may also refer to non-human species, such as mice, camels, goats, rabbits, or cows. A “subject” may be of any age, sex, and physical condition.

“Administering” a medicament to a patient refers to direct administration, which may be administration to a patient by a medical professional or may be self-administration, and/or indirect administration, which may be the act of prescribing a drug. For example, a physician who instructs a patient to self-administer a medicament or provides a patient with a prescription for a drug is administering the drug to the patient.

As used herein, the term “effective amount” of an agent, e.g., an anti-TfR1 single-domain antibody, is that amount sufficient to effect beneficial or desired results, for example, clinical results. As such, an “effective amount” depends upon the actual application. The term “effective amount” can be used interchangeably with “effective dose”, or “therapeutically effective amount”.

Complementarity determining region (CDR) refers to the hypervariable region in the variable region of an antibody molecule. Each heavy chain variable region or light chain variable region consists of three CDRs and four framework (FW) regions. These three CDR regions (CDR1, CDR2 and CDR3) together constitute the antigen binding site of the antibody.

### TfR1 Antigen Epitope Peptide

According to an aspect of the present disclosure, a human TfR1 antigen epitope peptide is provided, wherein the TfR1 antigen epitope peptide has an amino acid sequence selected from: (1) SEQ ID NO: 85; (2) SEQ ID NO: 97; (3) SEQ ID NO: 98; (4) SEQ ID NO: 100; and (5) SEQ ID NO: 101. With the TfR1 antigen epitope peptide, an anti-TfR1 antibody that specifically binds to the TfR1 protein with high affinity can be prepared.

In some embodiments, a TfR1 antigen is provided, which is a conjugate of the human TfR1 antigen epitope peptide as described above to a carrier protein.

In the present disclosure, the carrier protein includes, but is not limited to, keyhole limpet hemocyanin (KLH), bovine serum albumin (BSA) or chicken ovalbumin (OVA) protein. In some embodiments, the carrier protein is keyhole limpet hemocyanin (KLH) when the TfR1 antigen is used as an immunogen to immunize an animal to prepare antibodies. In addition, the carrier protein is bovine serum albumin (BSA) when the TfR1 antigen is used as a coating agent to screen monoclonal cell lines.

The use of the human TfR1 antigen epitope peptide or the TfR1 antigen as an immunogen to prepare anti-TfR1 antibodies also falls within the scope of protection of the present disclosure, wherein the use can be use for non-disease diagnosis or treatment purpose, or use for disease diagnosis or treatment purpose.

Therefore, the present disclosure also provides a human TfR1 antibody, which are produced using the TfR1 antigen as described above as an immunogen.

### Anti-TfR1 Single-Domain Antibody

According to an aspect of the present disclosure, an anti-TfR1 single-domain antibody is provided, the anti-TfR1 single-domain antibody including a heavy chain variable region, wherein the heavy chain variable region includes a heavy chain CDR1 as set forth in any one of SEQ ID NO: 53 to SEQ ID NO: 60, a heavy chain CDR2 as set forth in any one of SEQ ID NO: 61 to SEQ ID NO: 72, and a heavy chain CDR3 as set forth in any one of SEQ ID NO: 73 to SEQ ID NO: 82.

In some embodiments, sequences having at least about 60% homology, at least about 70% homology, at least about 75% homology, at least about 80% homology, at least about 85% homology, at least about 90% homology, at least about 95% homology, at least about 98% homology, or at least about 99% homology to the heavy chain CDR1 as set forth in SEQ ID NO: 53 to SEQ ID NO: 60, the heavy chain CDR2 as set forth in SEQ ID NO: 61 to SEQ ID NO: 72, and the heavy chain CDR3 as set forth in SEQ ID NO: 73 to SEQ ID NO: 82 can also achieve the purposes of the present disclosure.

In some embodiments, the heavy chain variable region further includes framework regions (FRs). The framework regions (FRs) includes FR1 as set forth in any one of SEQ ID NOS: 25-33, FR2 as set forth in any one of SEQ ID NOS: 34-40, FR3 as set forth in any one of SEQ ID NOS: 41-51, and FR4 as set forth in SEQ ID NO: 52.

In some embodiments, the framework region sequences FR1, FR2, FR3 and FR4 may also have conservative amino acid substitutions. In some embodiments, the anti-TfR1 single-domain antibody may include a FR1 having at least about 60%, about 65%, about 70%, about 75%, about 80%, about 85%, about 90%, about 95% sequence homology to any one of SEQ ID NOS: 25-33; a FR2 having at least about 60%, about 65%, about 70%, about 75%, about 80%, about 85%, about 90%, about 95% sequence homology to any one of SEQ ID NOS: 34-40; a FR3 having at least about 60%, about 70%, about 72%, about 74%, about 76%, about 78%, about 80%, about 82%, about 85%, about 88%, about 90%, about 92%, about 94%, about 96% sequence homology to any one of SEQ ID NOS: 41-51; and a FR4 having at least about 60%, about 65%, about 70%, about 75%, about 80%, about 85%, about 90% sequence homology to SEQ ID NOS: 52.

In some embodiments, the amino acid sequences of FR1, FR2, FR3 and FR4 have up to 10 amino acid substitutions, such as 1, 2, 3, 4, 5, 6, 7, 8, 9 or 10 conservative amino acid substitutions, relative to any one of SEQ ID NOS: 25-33, any one of SEQ ID NOS: 34-40, any one of SEQ ID NOS: 41-51 and SEQ ID NO: 52, respectively.

In some embodiments, the anti-TfR1 single-domain antibody of the present disclosure may be an Fc fusion antibody, a humanized antibody, a chimeric antibody, a monoclonal antibody, or a polyclonal antibody.

In some embodiments, a Fc fragment in the Fc fusion antibody is derived from IgG1, IgG2, IgG3, IgG4, IgM, IgA, IgD or IgE.

According to another aspect of the present disclosure, an anti-TfR1 single-domain antibody is provided, the amino acid sequence of the anti-TfR1 single-domain antibody being as set forth in any one of SEQ ID NOS: 1-12.

In some embodiments, sequences having at least about 80% homology, at least about 85% homology, at least about 90% homology, at least about 95% homology, at least about 98% homology, or at least about 99% homology to any one of SEQ ID NOS: 1-12 can also achieve the purposes of the present disclosure.

In some embodiments, sequences in which only one or several amino acids, for example, 1, 2, 3, 4, 5, 6, 7, 8, 9 or 10 conservative amino acid substitutions, are substituted relative to any one of SEQ ID NOS: 1-12 can also achieve the purposes of the present disclosure.

As used herein, the term “sequence homology” indicates the extent to which two (nucleotide or amino acid) sequences have identical residues at the same positions in an alignment, and is often expressed as a percentage. Preferably, homology is determined over the entire length of the sequences being compared. Thus, two copies of exactly the same sequence have 100% homology.

Indeed, in determining the degree of sequence homology between two amino acid sequences or in establishing the CDR1, CDR2 and CDR3 combination in the single-domain antibody, the skilled person may take into account “conservative” amino acid substitutions. In the case of substitutions, the substitutions are conservative amino acid substitutions. The conservative amino acid substitutions can generally be described as amino acid substitutions in which an amino acid residue is replaced with another amino acid residue of similar chemical structure and which has little or essentially no influence on the function, activity or other biological properties of the polypeptide. Such conservative amino acid substitutions are common in the art.

For example, the conservative amino acid substitutions are substitutions in which one or several amino acid within the following groups (a)-(d) is substituted by another one or several amino acid residue within the same group (a)-(d): (a) polar, negatively charged residues and their uncharged amides: Asp, Asn, Glu and Gln; (b) polar, positively charged residues: His, Arg and Lys; (c) aromatic residues: Phe, Trp and Tyr; and (d) nonpolar or weakly polar aliphatic residues: Ala, Ser, Thr, Gly, Pro, Met, Leu, Ile, Val, and Cys. In some embodiments, conservative amino acid substitutions are as follows: Asp into Glu; Asn into Gin, or into His; Glu into Asp; Gln into Asn; His into Asn, or into Gln; Arg into Lys; Lys into Arg, or into Gln; Phe into Met, into Leu, or into Tyr; Trp into Tyr; Tyr into Phe, or into Trp; Ala into Gly, or into Ser; Ser into Thr; Thr into Ser; Gly into Ala, or into Pro; Met into Leu, into Tyr, or into Ile; Leu into Ile, or into Val; Ile into Leu, or into Val; Val into Ile, or into Leu; and Cys into Ser.

In the present disclosure, the anti-TfR1 single-domain antibodies include not only complete single-domain antibodies, but also fragments, derivatives and analogs of anti-TfR1 single-domain antibodies. As used herein, the terms “fragment”, “derivative” and “analog” have the same meaning, and all refer to polypeptides that substantially maintain the same biological function or activity as the antibodies of the present disclosure. The fragment, derivative or analog of the polypeptides may be (i) one in which one or more of the conserved or non-conserved amino acid residues (particularly the conserved amino acid residues) are substituted and such substituted amino acid residue may or may not be one encoded by the genetic code, or (ii) one in which one or more of the amino acid residues has a substituent group, or (iii) one in which the mature polypeptides are fused with another compound, such as a compound to increase the half-life of the polypeptide (for example, polyethylene glycol), or (iv) one in which the additional amino acids are fused to such polypeptide sequences, such as a leader or secretory sequence or a sequence which is employed for purification of such polypeptides or fusion proteins formed with Fc tags. According to the teachings herein, these fragments, derivatives and analogs all fall within the scope of protection of the present disclosure.

The inventors of the present disclosure unexpectedly found that the anti-TfR1 single-domain antibody has a high affinity for TfR1 protein. Further, TfR1 is highly expressed on the cell surface of some disease tissues, especially on the cell surface of some cancer tissues. The inventors have verified that the use of the anti-TfR1 single-domain antibody disclosed in the present disclosure can induce an immune response in vivo, so the above-mentioned anti-TfR1 single-domain antibody can be used to effectively prevent or treat diseases associated with abnormal TfR1 receptor expression. For example, the disease is a tumor that abnormally expresses TfR1 receptors. For example, the tumor is breast cancer, glioma, lung adenocarcinoma, leukemia, lymphoma, non-Hodgkin's lymphoma or ovarian cancer. In view of the above, the anti-TfR1 single-domain antibody disclosed in the present disclosure can also be used to diagnose diseases associated with abnormal TfR1 receptor expression. For example, the disease is a tumor that abnormally expresses TfR1 receptors. For example, the tumor is breast cancer, glioma, lung adenocarcinoma, leukemia, lymphoma, non-Hodgkin's lymphoma or ovarian cancer.

In addition, the blood-brain barrier is like a mesh composed of endothelial cells, capillary basement membranes, astrocyte end feet and pericytes embedded in the capillary basement membrane. Many compounds in the blood (for example, all macromolecular drugs with a molecular weight greater than 1 kD and more than 98% of small molecule drugs) are almost difficult to cross the blood-brain barrier and enter the brain parenchyma network. The inventors also unexpectedly found that the expression level of TfR1 receptors in the blood-brain barrier is higher than that in normal tissues. Transferrin receptor TfR1 can mediate a transcellular transport mechanism. By using the anti-TfR1 single-domain antibody provided in the present disclosure, specific drugs can be effectively delivered across the blood-brain barrier to the brain parenchyma, thereby significantly improving the treatment of diseases, especially the treatment of nervous system diseases. For example, the nervous system diseases include, but are not limited to, Alzheimer's disease (AD), stroke, dementia, muscular dystrophy (MD), Parkinson's disease, traumatic brain injury, etc.

The anti-TfR1 single-domain antibody in the present disclosure specifically targets TfR1 protein, but has low immunogenicity, and thus does not produce adverse side effects in the subject. The anti-TfR1 single-domain antibody has good compatibility in the subject, especially humans, and can be advantageously used as a carrier for delivering drugs or directly used as a drug.

In addition, the anti-TfR1 antibody in the present disclosure is a single-domain antibody, which has a small molecular size and a simple structure, and is therefore easy to express in a variety of expression systems. For example, it can be expressed in both prokaryotic systems and eukaryotic systems such as yeast cells or mammalian cells. Therefore, it can be prepared at a lower production cost and is suitable for industrial large-scale production.

### Recombinant Protein

According to an aspect of the present disclosure, a recombinant protein is provided, including the amino acid sequence of the anti-TfR1 single-domain antibody as described above.

In some embodiments, the recombinant protein may include another amino acid sequence in addition to the amino acid sequence of the anti-TfR1 single-domain antibody as described above. For example, the another amino acid sequence may include a leader sequence, a linker sequence, a tag sequence, a green fluorescent protein sequence, or the like.

In some embodiments, the recombinant protein can be a multi-epitope antibody, a multivalent antibody, or a multispecific antibody.

In some embodiments, the recombinant protein can be a multi-epitope antibody. For example, the multi-epitope antibody includes a plurality of sequences selected from SEQ ID NOS: 1 to 12. In one embodiment, any one sequence in the multi-epitope antibody is directly linked to another adjacent sequence. In another embodiment, any two adjacent sequences in the multi-epitope antibody can be linked via a linker sequence. In another embodiment, the multi-epitope antibody consists of a plurality of sequences selected from SEQ ID NOS: 1 to 12. In other embodiments, the multi-epitope antibody has 2 to 10 different epitopes, for example 2, 3, 4, 5, 6, 7, 8, 9 or 10 different epitopes.

In some embodiments, the recombinant protein is a multivalent antibody. In some embodiments, the multivalent antibody includes several repeats of any one of SEQ ID NOS: 1 to 12. In some embodiments, the repeats are linked together directly or via a linker sequence. In some embodiments, the multivalent antibody consists of several repeats of any one of SEQ ID NOS: 1-12. In some embodiments, the multivalent antibody has 2 to 20 repeats, such as 2, 3, 4, 5, 6, 7, 8, 9, 10, 11, 12, 13, 14, 15, 16, 17, 18, 19 or 20 repeats.

In some embodiments, the recombinant protein can be a multispecific antibody. In some embodiments, the multispecific antibody includes but is not limited to a bispecific antibody, a trispecific antibody, a tetraspecific antibody or a pentaspecific antibody. Typically, the multispecific antibody is a bispecific antibody. For example, in addition to targeting the TfR1 antigen, the multispecific antibody can also target other antigens, such as PD-1, PD-L1, CD19, CD22, EGFR, etc.

In the present disclosure, the anti-TfR1 single-domain antibody has a small molecular size and a simple structure, and is therefore easy to be engineered to obtain multi-epitope antibodies, multivalent antibodies or multispecific antibodies, etc., thereby expanding the application scope of the anti-TfR1 single-domain antibody.

### Antibody Complex

According to another aspect of the present disclosure, an antibody complex is provided, including the anti-TfR1 single-domain antibody as described above or the recombinant protein as described above.

In some embodiments, the antibody complex can be a conjugate formed by conjugating the anti-TfR1 single-domain antibody as described above or the recombinant protein as described above to an active ingredient selected from a therapeutic agent, a diagnostic agent, a carrier protein, a liposome or the like.

In some embodiments, the anti-TfR1 single-domain antibody as described above or the recombinant protein as described above can be conjugated to the therapeutic agent, the diagnostic agent or the carrier protein via a linker. The linker has a sequence of 5 to 30 amino acids, such as 5 to 25 amino acids, 5 to 20 amino acids, 5 to 15 amino acids, or 5 to 10 amino acids.

In some embodiments, the anti-TfR1 single-domain antibody as described above or the recombinant protein as described above can be conjugated to the liposome via a covalent bond or a non-covalent bond. In some embodiments, the covalent bond conjugating includes conjugating to the liposome via a disulfide bond on the anti-TfR1 single-domain antibody or the recombinant protein, or conjugating to the anti-TfR1 single-domain antibody or the recombinant protein via a hydrophobic group on the liposome.

In some embodiments, the therapeutic agent may include, but is not limited to, cisplatin, paclitaxel, cyclophosphamide, doxorubicin, daunorubicin, idarubicin, gemcitabine, interferons (e.g., interferon-α, interferon-β, interferon-γ), interleukins (e.g., interleukin-1 (IL-1), IL-2, IL-3, IL-6, IL-10, IL-12, IL-18, and IL-21), tumor necrosis factor (TNF) (e.g., TNFα), granulocyte colony stimulating factor (G-CSF), and granulocyte macrophage colony stimulating factor (GM-CSF), or the like.

In some embodiments, the carrier protein may include, but is not limited to, keyhole limpet hemocyanin (KLH), serum albumin (e.g., bovine serum albumin (BSA)) or ovalbumin, immunoglobulin Fc domain, tetanus toxoid, diphtheria toxoid, or a combination thereof.

In some embodiments, the diagnostic agent can include an imaging agent or a label. Useful labels include magnetic beads, fluorescent dyes (e.g., fluorescein isothiocyanate, Texas Red, rhodamine, green fluorescent protein, etc.), radioactive labels, enzymes (e.g., horseradish peroxidase, alkaline phosphatase, and other enzymes commonly used in ELISA), colorimetric labels (such as colloidal gold, colored glass, plastic beads, or nanoparticles (e.g., polystyrene, polypropylene, latex, etc.)), etc.

The present disclosure can provide a conjugate of an anti-TfR1 single-domain antibody or a recombinant protein thereof to a therapeutic agent. Thus, based on the transcellular transport mechanism mediated by transferrin receptor TfR1, the anti-TfR1 single-domain antibody described herein can be used to effectively deliver the therapeutic agent to the target site of interest.

In some embodiments, the antibody complex can cross the blood-brain barrier and deliver the therapeutic agent to the brain parenchyma, thereby significantly improving the treatment of brain diseases, especially the treatment of nervous system diseases. For example, the nervous system diseases include, but are not limited to, Alzheimer's disease (AD), stroke, dementia, muscular dystrophy (MD), Parkinson's disease, traumatic brain injury, etc.

In some embodiments, the antibody complex can also deliver the therapeutic agent to the tumor tissue based on the transcellular transport mechanism mediated by transferrin receptor TfR1, thereby significantly enhancing the ability to kill the tumor.

Similarly, the present disclosure may also provide a conjugate of an anti-TfR1 single-domain antibody or a recombinant protein thereof to a diagnostic agent. Thus, the anti-TfR1 single-domain antibody conjugated to a diagnostic agent can across the blood-brain barrier and be delivered to the target site, and thus used to diagnose a disease.

Therefore, in another aspect, the present disclosure provides a method for transporting a drug across the blood-brain barrier, including exposing the anti-TfR1 single-domain antibody conjugated to drug of the present disclosure to the blood-brain barrier, so that the anti-TfR1 single-domain antibody transports the drug conjugated thereto across the blood-brain barrier to achieve specific therapeutic or diagnostic purposes as well as non-therapeutic or diagnostic purposes. The above-mentioned drug can be a therapeutic agent or a diagnostic agent.

### Nucleic Acids

According to another aspect of the present disclosure, a nucleic acid is provided, the nucleic acid encoding the anti-TfR1 single-domain antibody as described above or the recombinant protein as described above.

In some embodiments, the nucleic acid has a nucleotide sequence as set forth in any one of SEQ ID NOS: 13-24.

In some embodiments, nucleotide sequences having at least about 80% homology, for example at least about 85% homology, at least about 90% homology, at least about 95% homology, at least about 98% homology, or at least about 99% homology to any one of SEQ ID NOS: 1-12 can also achieve the purposes of the present disclosure. The anti-TfR1 single-domain antibody encoded by the nucleic acid can specifically bind to the TfR1 protein.

In some embodiments, the nucleic acid can be used as a drug. The nucleic acid can accurately express the anti-TfR1 single-domain antibody in a subject, and thus can be used to prevent or treat diseases associated with abnormal TfR1 receptor expression.

### Expression Vector

According to another aspect of the present disclosure, an expression vector is provided, including the nucleic acid as described above. The expression vector expresses the anti-TfR1 single-domain antibody or recombinant protein thereof as described in the present disclosure.

In one embodiment, the expression vector is obtained by integrating the nucleic acid encoding the anti-TfR1 single-domain antibody or recombinant protein thereof into a vector RJK-V4-hFC1 by genetic engineering means. The RJK-V4-hFC1 is prepared by Example 10 of the present disclosure.

By using the expression vector in the present disclosure, anti-TfR1 single-domain antibody or recombinant protein thereof can be accurately and stably expressed.

### Host Cell

According to another aspect of the present disclosure, a host cell is provided, including the expression vector as described in the present disclosure. The host cell expresses the anti-TfR1 single-domain antibody or recombinant protein as described in the present disclosure.

In some embodiments, the host cell is a bacterial cell, a fungal cell, or a mammalian cell.

In some embodiments, the host cell includes a prokaryotic cell or a eukaryotic cell, including bacteria, or fungi.

In some embodiments, the host cell is selected from the group consisting of Escherichia coli, a yeast cell, a mammalian cell, bacteriophage, or a combination thereof.

In some embodiments, the prokaryotic cell is selected from the group consisting of Escherichia coli, Bacillus subtilis, Lactobacillus, Streptomyces, Proteus mirabilis, or a combination thereof.

In some embodiments, the eukaryotic cell is selected from the group consisting of Pichia pastoris, Saccharomyces cerevisiae, Schizosaccharomyces pombe, Trichoderma, or a combination thereof.

In some embodiments, the eukaryotic cell is selected from the group consisting of an cell from an insect such as fall armyworm, a cell from a plant such as tobacco, a Syrian baby hamster kidney cell (BHK cell), a Chinese hamster ovary cell (CHO cell), an African green monkey kidney cell (COS cell), a myeloma cell, or a combination thereof.

In additional embodiments, the host cell is a suspension ExpiCHO-S cell.

In other embodiments, the host cell is a suspension human embryonic kidney epithelial 293F cell.

The anti-TfR1 antibody in the present disclosure is a single-domain antibody, which has a small molecular size and a simple structure, and is therefore easy to express in a variety of expression systems. For example, it can be expressed in both prokaryotic systems and eukaryotic systems such as yeast cells or mammalian cells. Therefore, it can be prepared at a lower production cost.

### Pharmaceutical Composition

According to another aspect of the present disclosure, a pharmaceutical composition is provided, including the anti-TfR1 single-domain antibody, the recombinant protein thereof, the antibody complex thereof, the nucleic acid encoding therefor, the expression vector expressing therefor, or the host cell expressing therefor as an active ingredient, and a pharmaceutically acceptable carrier.

In some embodiments, the pharmaceutical composition includes the anti-TfR1 single-domain antibody, the recombinant protein thereof, or the antibody complex thereof as an active ingredient, and a pharmaceutically acceptable carrier

In another embodiment, the pharmaceutical composition includes the anti-TfR1 single-domain antibody as an active ingredient, and a pharmaceutically acceptable carrier.

Typically, the anti-TfR1 single-domain antibody can be formulated in a non-toxic, inert and pharmaceutically acceptable aqueous carrier. The pH is generally determined according to the isoelectric point of the anti-TfR1 single-domain antibody. The pH of the aqueous carrier needs to deviate from the isoelectric point of the anti-TfR1 single-domain antibody and differ from the isoelectric point of the anti-TfR1 single-domain antibody by about 2.

The pharmaceutical composition of the present disclosure contains a safe and effective amount, for example, 0.001 wt % to 99 wt %, 0.01 wt % to 90 wt %, or 0.1 wt % to 80 wt % of the anti-TfR1 single-domain antibody, recombinant protein thereof, or antibody complex thereof, and a pharmaceutically acceptable carrier.

In some embodiments, the carrier includes but is not limited to, saline, buffer, glucose, water, glycerol, ethanol, or combinations thereof.

The pharmaceutical preparation should match the mode of administration. The pharmaceutical composition in the present disclosure can be prepared in the form of an injection, for example, by conventional methods using physiological saline or an aqueous solution containing glucose and other adjuvants. The dosage form of the pharmaceutical composition such as an injection or solution is preferably manufactured under sterile conditions.

As described herein, the anti-TfR1 single-domain antibody, recombinant protein thereof, antibody complex thereof, or a pharmaceutical composition including the same can be administered parenterally, topically, intravenously, orally, intragastrically, subcutaneously, intraarterially, intracranially, intraperitoneally, intranasally, or intramuscularly for therapeutic and/or prophylactic treatment. In some embodiments, the anti-TfR1 single-domain antibody, recombinant protein thereof, antibody complex thereof, or a pharmaceutical composition including the same is administered to a subject by intramuscular injection or intravenous injection.

According to an embodiment of the present disclosure, the pharmaceutical composition as described above can be used to effectively prevent or treat diseases associated with abnormal TfR1 receptor expression. For example, the disease is a tumor that abnormally expresses TfR1 receptors. For example, the tumor is breast cancer, glioma, lung adenocarcinoma, leukemia, lymphoma, non-Hodgkin's lymphoma or ovarian cancer.

In addition, according to the embodiments of the present disclosure, transferrin receptor TfR1 can mediate a transcellular transport mechanism. By using the pharmaceutical composition provided in the present disclosure, specific drugs can be effectively delivered across the blood-brain barrier to the brain parenchyma, thereby significantly improving the treatment of diseases, especially the treatment of nervous system diseases. For example, the nervous system diseases include, but are not limited to, Alzheimer's disease (AD), stroke, dementia, muscular dystrophy (MD), Parkinson's disease, traumatic brain injury, etc.

### Kit

In another aspect of the present disclosure, a kit is provided, including the anti-TfR1 single-domain antibody, recombinant protein, antibody complex, nucleic acid, expression vector, host cell or pharmaceutical composition as described in the present disclosure.

In some embodiments, the kit further includes one or more containers, or instruction for use. The anti-TfR1 single-domain antibody, the recombinant protein, the antibody complex, the nucleic acid, the expression vector, the host cell or the pharmaceutical composition may be contained in one or more containers to facilitate administration to a subject. The one or more containers may also be used to accommodate additional agents for preventing or treating diseases. The kit may also include a device for delivering the active ingredients in the kit, such as a needle or a syringe. The instructions for use indicate information such as the administration mode, dosage, side effects, etc. for preventing or treating diseases associated with abnormal TfR1 receptor expression.

According to an embodiment of the present disclosure, the kit can be used to prevent or treat a disease associated with abnormal TfR1 receptor expression, such as a tumor that abnormally expresses TfR1 receptors. For example, the tumor is breast cancer, glioma, lung adenocarcinoma, leukemia, lymphoma, non-Hodgkin's lymphoma or ovarian cancer. The kit can also be used to prevent or treat brain diseases, such as nervous system diseases. The anti-TfR1 single-domain antibody is used to transport the therapeutic agent across the blood-brain barrier to achieve the therapeutic purpose.

In some embodiments, the kit is used for detecting TfR1 protein in vitro. The kit can be an in vitro detection device used for detecting the level of TfR1 protein.

In some embodiments, the kit includes the anti-TfR1 single-domain antibody, a lysis medium for dissolving the sample, and a universal reagent for detection (e.g., a detection label such as an enzyme or a fluorescent or radioactive label, a detection substrate, a buffer, etc.). In one embodiment, the kit may also include an antibody against the anti-TfR1 single-domain antibody (i.e., an anti-antibody) as a secondary antibody.

According to an embodiment of the present disclosure, the kit can be used to diagnose a disease associated with abnormal TfR1 receptor expression, with high sensitivity and specificity.

### Use of Anti-TfR1 Single-Domain Antibody

In yet another aspect of the present disclosure, use of the anti-TfR1 single-domain antibody, the recombinant protein, the antibody complex, the nucleic acid, the expression vector, the host cell, or the pharmaceutical composition in manufacturing a medicament for preventing or treating a disease associated with abnormal TfR1 receptor expression is provided.

In yet another aspect of the present disclosure, an anti-TfR1 single-domain antibody, a recombinant protein, an antibody complex, a nucleic acid, an expression vector, a host cell, or a pharmaceutical composition for use as a drug is provided.

In yet another aspect of the present disclosure, an anti-TfR1 single-domain antibody, a recombinant protein, an antibody complex, a nucleic acid, an expression vector, a host cell, or a pharmaceutical composition for use in preventing or treating a disease associated with abnormal TfR1 receptor expression is provided.

In some embodiments, the disease associated with abnormal TfR1 receptor expression is a tumor associated with abnormal TfR1 receptor expression. In some embodiments, the tumor includes but is not limited to breast cancer, glioma, lung adenocarcinoma, leukemia, lymphoma, non-Hodgkin's lymphoma, ovarian cancer, or the like. By using the anti-TfR1 single-domain antibody and the recombinant protein, antibody complex or pharmaceutical composition thereof, the diseases can be effectively prevented or treated.

In yet another aspect of the present disclosure, an anti-TfR1 single-domain antibody, a recombinant protein, an antibody complex, or the pharmaceutical composition for use as a drug for crossing the blood-brain barrier is provided.

The inventors also unexpectedly found that the expression level of TfR1 receptors in the blood-brain barrier is higher than that in normal tissues. Transferrin receptor TfR1 can mediate a transcellular transport mechanism. By using the anti-TfR1 single-domain antibody provided in the present disclosure, specific drugs can be effectively delivered across the blood-brain barrier to the brain parenchyma, thereby significantly improving the treatment of diseases, especially the treatment of nervous system diseases. For example, the nervous system diseases include, but are not limited to, Alzheimer's disease (AD), stroke, dementia, muscular dystrophy (MD), Parkinson's disease, traumatic brain injury, etc.

### Method for Preventing or Treating Diseases

In yet another aspect of the present disclosure, a method for preventing or treating a disease associated with abnormal TfR1 receptor expression, including administering to a subject in need thereof a therapeutically effective amount of the anti-TfR1 single-domain antibody, the recombinant protein, the antibody complex, the nucleic acid, the expression vector, the host cell, or the pharmaceutical composition.

In some embodiments, the disease associated with abnormal TfR1 receptor expression is a tumor associated with abnormal TfR1 receptor expression. In some embodiments, the tumor includes but is not limited to breast cancer, glioma, lung adenocarcinoma, leukemia, lymphoma, non-Hodgkin's lymphoma, ovarian cancer, or the like. By using the anti-TfR1 single-domain antibody, the diseases can be effectively prevented or treated.

In yet another aspect of the present disclosure, a method for transporting a drug across the blood-brain barrier, including exposing the anti-TfR1 single-domain antibody conjugated to drug of the present disclosure (i.e., the antibody complex of the present disclosure) to the blood-brain barrier, so that the anti-TfR1 single-domain antibody transports the drug conjugated thereto across the blood-brain barrier to achieve a specific preventive or therapeutic purpose. The above method is used to prevent or treat brain diseases, especially nervous system diseases. For example, the nervous system diseases include, but are not limited to, Alzheimer's disease (AD), stroke, dementia, muscular dystrophy (MD), Parkinson's disease, traumatic brain injury, etc.

### Method and System for Diagnosing Diseases

In yet another aspect of the present disclosure, a method for diagnosing a disease associated with abnormal TfR1 receptor expression is provided. According to an embodiment of the present disclosure, the diagnostic method includes contacting the anti-TfR1 single-domain antibody as described above with a biological sample from a subject to detect an expression level of TfR1 protein in the biological sample, and comparing the detected expression level value of the TfR1 protein with a normal expression level value of the TfR1 protein in the same biological sample from a normal subject. The detected expression level value being higher than the normal expression level value indicates that the subject is at a risk of suffering from a disease associated with abnormal TfR1 receptor expression.

In some embodiments, the disease is a tumor that abnormally expresses the TfR1 receptor. In some embodiments, the tumor is breast cancer, glioma, lung adenocarcinoma, leukemia, lymphoma, non-Hodgkin's lymphoma or ovarian cancer.

In yet another aspect of the present disclosure, a system for diagnosis is provided. The diagnostic system includes: a detection device; and a diagnostic result determination device. The detection device is configured to contact the anti-TfR1 single-domain antibody as described above with a biological sample from a subject to detect an expression level of the TfR1 protein in the biological sample. The diagnostic result determination device is connected to the detection device and is configured to compare the detected expression level value of the TfR1 protein with a normal expression level value of the TfR1 protein in the same biological sample from a normal subject, wherein the detected expression level value being higher than the normal expression level value indicates that the subject is at a risk of suffering from a disease associated with abnormal TfR1 receptor expression.

### Preparation Method

In an embodiment of the present disclosure, a method for producing an anti-TfR1 single-domain antibody is provided, including: (a) culturing the host cell as described above under conditions suitable for producing the single-domain antibody to obtain a culture containing the anti-TfR1 single-domain antibody; (b) isolating the anti-TfR1 single-domain antibody from the culture; and optionally (c) purifying and/or modifying the anti-TfR1 single-domain antibody obtained in (b).

In another embodiment of the present disclosure, a method for preparing an anti-TfR1 single-domain antibody includes: immunizing a camel with TfR1 protein or a truncated form thereof as an immunogen to produce the anti-TfR1 single-domain antibody; harvesting gene fragments of the anti-TfR1 single-domain antibody from immunized peripheral blood lymphocytes or spleen cells; recombining the above gene fragments into a vector for phage display; screening for the anti-TfR1 single-domain antibody against TfR1 protein; and isolating and purifying the above anti-TfR1 single-domain antibodies.

The embodiments of the present disclosure are described in detail below. It should be noted that the embodiments described herein are exemplary only, and are intended to explain the present invention, but should not be construed as limiting the present disclosure. Furthermore, unless stated otherwise, all reagents utilized in the following examples are commercially available or can be synthesized using methods herein or known in the art. The reaction conditions that are not stated can be easily obtained by those skilled in the art.

### Example 1: Preparation of Humanized TfR1 Protein

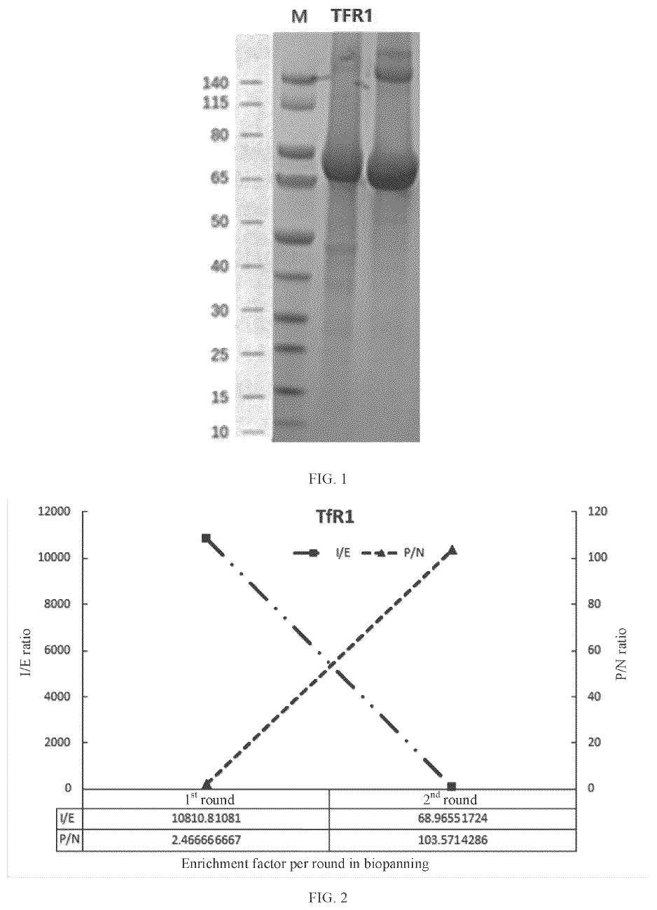

In this example, the human recombinant TfR1 protein used was obtained through expression and purification by Nanjing Regenecore Biotech Co., Ltd. The expression vector for this humanized recombinant TfR1 protein was designed according to the following protocol:
(1) The coding sequence of TfR1 protein (Accession No.: NM_001128148.2) was retrieved from NCBI, encoding for an amino acid sequence with Accession No. NP_001121620.1.
(2) The coding sequence encoding amino acids 89-760 of TfR1 protein was cloned into pcDNA3.4 vector (from Invitrogen) through gene synthesis. The constructed vector was subjected to Sanger sequencing by Genewiz, and the sequence obtained by sequencing was aligned with the original sequence to confirm the successful acquisition of the correct recombinant plasmid. Subsequently, this recombinant plasmid underwent maxi-prep and endotoxin removal, followed by transfection into suspension 293F cells for TfR1 protein expression and purification. The obtained humanized TfR1 protein achieved a purity of over 90%, which met the requirements for animal immunization. SDS-PAGE electrophoretogram of the purified humanized TfR1 protein is shown in FIG. 1.

### Example 2: Construction of Libraries of Anti-Humanized TfR1 Protein Single-Domain Antibodies

1 mg of the humanized recombinant TfR1 protein obtained in Example 1 was mixed with an equal volume of Freund's complete adjuvant (purchased from Sigma-Aldrich, Cat. No. F5881-6X10ML) for immunizing Alxa Bactrian camels from Inner Mongolia. Immunization was performed once per week for a total of seven consecutive times. For all immunizations except the first, 1 mg of TfR1 protein was mixed with an equal volume of Freund's incomplete adjuvant (purchased from Sigma-Aldrich) to immunize the animals. This immunization process was used to stimulate the Bactrian camels to produce antibodies against the TfR1 protein.

After immunization, 150 mL of peripheral blood lymphocytes were collected from the Bactrian camels to extract RNA of the peripheral blood lymphocytes. The extracted total RNA was used to synthesize cDNA, and VHH fragments (i.e., the variable heavy chain regions of antibodies) were amplified from the cDNA as template via nested PCR. The VHH fragments and the pMECS vector were digested separately with restriction endonucleases (PstI, Cat. No. R3140L; NotI, Cat. No. R3189L; BstEII, Cat. No. R3162S; all purchased from NEB (New England BioLabs)). The digested fragments and vector were ligated, and the vectors ligated with the fragments were electrotransformed into competent TG1 cells (purchased from Agilent). A phage display library against TfR1 protein was constructed and determined for the library size, which was approximately 1×10⁹ Meanwhile, colony PCR identification was performed to assess the correct insertion rate of the target fragments in the library.

The results showed that among 30 clones randomly selected from the library and subjected to PCR amplification, 28 clones produced bands of the predicted size, while 2 clones exhibited incorrect amplification bands, yielding a correct insertion rate of approximately 93%.

### Example 3: Screening of Anti-Humanized TfR1 Protein Single-Domain Antibodies

200 μL of recombinant TG1 cells obtained in Example 2 were cultured in 2×TY medium (purchased from Thermo Fisher). During cultivation, 40 μL of helper phage VCSM13 (purchased from Agilent) was added to infect the TG1 cells, followed by overnight incubation to amplify the phages. The next day, the amplified phages were precipitated with PEG/NaCl, centrifuged, and collected.

500 ng of TfR1 protein was diluted in 100 μL of 100 mM NaHCO₃ (pH 8.3). The TfR1 protein mixture was added to the wells of an ELISA plate and incubated overnight at 4° C. to coat the TfR1 protein onto the well surfaces. Negative control wells (i.e., medium controls) were also established. The following day, 200 μL of 3% skim milk was added to block at room temperature for 2 hours. After blocking, 100 μL of the amplified phage library (approximately 2×10¹¹ phage particles) was added and reacted at room temperature for 1 hour. The wells were then washed 15 times with PBS containing 0.05% Tween-20 to remove unbound phage particles.

Phage particles specifically bound to TfR1 protein were eluted using trypsin at a final concentration of 25 mg/mL. The phages were used to infect log-phase E. coli TG1 cells, followed by incubation at 37° C. for 1 hour, and the phages were produced and collected for the next round of screening. The same screening process was repeated once to achieve gradual enrichment. Based on this screening protocol, two rounds of screening were performed for anti-humanized TfR1 protein single-domain antibodies. The screening was terminated when the enrichment factor of anti-humanized TfR 1 protein phage reached over 10. The enrichment effects are shown in FIG. 2.

In FIG. 2, P/N=(number of monoclonal bacterial colonies grown from TG1 cells infected with phage eluted from positive wells in biopanning)/(number of monoclonal bacterial colonies grown from TG1 cells infected with phage eluted from negative wells in biopanning). The parameter P/N progressively increased after enrichment. I/E=(total phage input per round in positive wells in biopanning)/(total phage eluted per round from positive wells in biopanning). The parameter I/E progressively approached 1 after enrichment.

This example demonstrated effective enrichment of anti-humanized TfR1 protein single-domain antibodies after two rounds of solid-phase panning. In the second round, P/N reached 103.57 and I/E reached 68.96, indicating good enrichment effects.

### Example 4: Screening of Specific Positive Clones Against Humanized TfR1 Protein by Phage Enzyme-Linked Immunosorbent Assay (ELISA)

384 single colonies from the positive clones obtained via the screening method described in Example 3 were inoculated into a 96-deep-well plate containing 2×TY medium with 100 g/mL ampicillin and, along with blank controls, cultured at 37° C. until the log phase was reached. Subsequently, isopropyl β-D-1-thiogalactopyranoside (IPTG) (purchased from Sangon Biotech, Cat. No. A600168-0100) was added to a final concentration of 1 mM, followed by overnight incubation at 28° C.

100 ng of humanized TfR 1 protein was diluted in 100 μL of 100 mM NaHCO₃ (pH 8.3). The mixture containing TfR1 protein was added to the wells of an ELISA plate and incubated overnight at 4° C. to coat the protein onto the well surfaces.

Crude antibody extracts were obtained using osmotic lysis. 100 μL of the obtained crude antibody extract was transferred to the TfR1-coated ELISA plate and incubated at room temperature for 1 hour. Unbound antibodies were washed away with PBST buffer with Tween-20 (PBS purchased from Sangon Biotech, Cat. No. B548117-0500; Tween-20 purchased from Beyotime Biotechnology, Cat. No. ST825). 100 μL of 1:2000-diluted mouse anti-HA horseradish peroxidase (HRP)-conjugated antibody (Thermo Fisher) was added and incubated at room temperature for 1 hour. After washing with PBST buffer to remove unbound antibodies, HRP chromogenic solution was added, followed by a 15-minute reaction at 37° C. With the stop solution added, the absorbance was read at 450 nm using a microplate reader (purchased from TECAN).

A well was identified as a positive clone well if its OD value was at least 5-fold higher than that of the control well. The bacterial solution from positive clone wells were transferred to LB medium containing 100 μg/mL ampicillin and cultured with shaking. Plasmids were then extracted and subjected to sequencing.

Gene sequences of individual clones were analyzed using the VectorNTI sequence alignment software. Clones with identical CDR1, CDR2, and CDR3 sequences were regarded as the same clone, while those with distinct sequences were regarded as different clones. Ultimately, specific anti-humanized TfR1 protein single-domain antibodies were obtained (including single-domain antibody clones having sequences unlisted, etc.).

The anti-humanized TfR1 protein single-domain antibodies exhibited an FR1-CDR1-FR2-CDR2-FR3-CDR3-FR4 structure. The harvested recombinant plasmids can be expressed in a prokaryotic system to produce anti-humanized TfR1 protein single-domain antibodies.

Twelve anti-humanized TfR1 protein single-domain antibodies (designated as 2H7, 4C5, 4D11, 5A3, 5D1, 5D3, 7A3, 7D10, 7G10, 7G5, 8B8, and 8E10) were obtained, and have amino acid sequences as set forth in SEQ ID NOS: 1-12 respectively, and nucleotide sequences as set forth in SEQ ID NOS: 13-24 respectively.

The CDR1, CDR2, and CDR3 sequences of the twelve anti-TfR1 single-domain antibodies are shown in Tables 1-3, respectively. These CDR sequences were determined according to the IMGT numbering rules. Engineering involving the Fc region were performed based on the EU numbering rules.

TABLE 1
CDR1 sequences of 12 single-domain antibodies

| Actual clone number | CDR1 | SEQ ID |
|---|---|---|
| 7D10 | GGTFEMYA | SEQ ID NO: 53 |
| 5D3 | GNIFSINN | SEQ ID NO: 54 |
| 8E10 | GPPFSTYG | SEQ ID NO: 55 |
| 7G10 | GRTFISYA | SEQ ID NO: 56 |
| 8B8 | GRTFSTSG | SEQ ID NO: 57 |
| 7G5 | GSIFSIDT | SEQ ID NO: 58 |
| 2H7 | GSIWSIYT | SEQ ID NO: 59 |
| 4C5 | | |
| 4D11 | | |
| 5D1 | | |
| 7A3 | | |
| 5A3 | RRAFNTKA | SEQ ID NO: 60 |

TABLE 2
CDR2 sequences of 12 single-domain antibodies

| Actual clone number | CDR2 | SEQ ID |
|---|---|---|
| 7G10 | ERWSGSGI | SEQ ID NO: 61 |
| 7D10 | FSWRDSSTT | SEQ ID NO: 62 |
| 4C5 | IAREGGR | SEQ ID NO: 63 |
| 8E10 | ISGSGFTT | SEQ ID NO: 64 |
| 5A3 | ISWSGFST | SEQ ID NO: 65 |
| 8B8 | ISWSGFTT | SEQ ID NO: 66 |
| 2H7 | ITGDST | SEQ ID NO: 67 |
| 7G5 | ITKGGSR | SEQ ID NO: 68 |
| 5D1 | ITRDGGR | SEQ ID NO: 69 |
| 7A3 | ITRDSGR | SEQ ID NO: 70 |
| 4D11 | ITRDST | SEQ ID NO: 71 |
| 5D3 | ITRGGIT | SEQ ID NO: 72 |

TABLE 3
CDR3 sequences of 12 single-domain antibodies

| Actual clone | | |
|---|---|---|
| number | CDR3 | SEQ ID |
| 5A3 | AAGVLDGTNWHEYDY | SEQ ID NO: 73 |
| 8E10 | | |
| 8B8 | AAGVLDSTSWHEYDY | SEQ ID NO: 74 |
| 7G10 | AAREGGLLHVATSYDY | SEQ ID NO: 75 |
| 7D10 | AARPYVSRWSESSGYDY | SEQ ID NO: 76 |
| 4D11 | HAITSLGEY | SEQ ID NO: 77 |
| 7A3 | | |
| 7G5 | LADRLFRDRS | SEQ ID NO: 78 |
| 5D1 | LAITSLGEY | SEQ ID NO: 79 |
| 5D3 | NAHPWRLGIADY | SEQ ID NO: 80 |
| 4C5 | NAITSLGEY | SEQ ID NO: 81 |
| 2H7 | QAITSLGEY | SEQ ID NO: 82 |

The FR1, FR2, FR3, and FR4 sequences of the twelve anti-TfR1 single-domain antibodies are shown in Tables 4-7, respectively. These FR sequences were determined according to the numbering scheme based on the “IMGT numbering rules.”.

TABLE 4
FR1 sequences of 12 single-domain antibodies

| Actual | | |
|---|---|---|
| clone | | |
| number | FR1 | SEQ ID |
| 5D3 | ESGGGLVEPGGSLRLSCAAS | SEQ ID NO: 25 |
| 7G10 | ESGGGLVQAGASLKLSCNGS | SEQ ID NO: 26 |
| 8E10 | ESGGGLVQAGGSLRLSCAFS | SEQ ID NO: 27 |
| 5A3 | ESGGGLVQAGGSLTLSCAVS | SEQ ID NO: 28 |
| 4C5 | ESGGGLVQPGESLRLSCAAS | SEQ ID NO: 29 |
| 2H7 | ESGGGLVQPGESLRLSCVAS | SEQ ID NO: 30 |
| 4D11 | | |
| 5D1 | | |
| 7A3 | | |
| 7D10 | ESGGGLVQPGGSLRLSCATS | SEQ ID NO: 31 |
| 8B8 | ESGGGLVQPGGSLRLSCVAS | SEQ ID NO: 32 |
| 7G5 | ESGGGSVQPGGSLRLSCAVS | SEQ ID NO: 33 |

TABLE 5
FR2 sequences of 12 single-domain antibodies

| Actual clone | | |
|---|---|---|
| number | FR2 | SEQ ID |
| 7D10 | MAWFRQAPGKDREGVAG | SEQ ID NO: 34 |
| 7G5 | MAWYRQAPGKQRELVAA | SEQ ID NO: 35 |
| 5A3 | MGWFRQAPGKEREFVAT | SEQ ID NO: 36 |
| 8B8 | | |
| 8E10 | | |
| 7G10 | MGWFRQAPGKQREFVAA | SEQ ID NO: 37 |
| 2H7 | MGWYRQAPGKQRDFVAS | SEQ ID NO: 38 |
| 4D11 | | |
| 5D1 | | |
| 7A3 | | |
| 4C5 | MGWYRQAPGLQRDFVGS | SEQ ID NO: 39 |
| 5D3 | MYWHRQAPGKQRELVGG | SEQ ID NO: 40 |

TABLE 6
FR3 sequences of 12 single-domain antibodies

| Actual | | |
|---|---|---|
| clone | | |
| number | FR3 | SEQ ID |
| 7G5 | NYADSVKGRFTISRDNAKNTVYLQMNSLKPEDTAVYYC | SEQ ID NO: 41 |
| 5A3 | NYADSVKGRFTISRDNAKNTVYLQMNSLKPGDTAVYYC | SEQ ID NO: 42 |
| 5D3 | NYVDSVKGRFTISRDNAKNTVYLQMNSLKPEDTAVYFC | SEQ ID NO: 43 |
| 5D1 | RHAESVKDRFTISRDNAKNTLYLQMNSLKPEDTAVYSC | SEQ ID NO: 44 |
| 4D11 | RTLESVKDRFIISRDNAKNTLYLQMNSLKPEDTAVYYC | SEQ ID NO: 45 |
| 2H7 | RTLESVKDRFIISRDNTKNTLYLQMNSLKPEDTAVYYC | SEQ ID NO: 46 |
| 7A3 | RYAESVKDRFTISRDNAKNTLYLQTNSLKPEDTAVYYC | SEQ ID NO: 47 |
| 4C5 | RYAESVKDRFTISRDNAKNTMYLQMNSLKREDTAVYYC | SEQ ID NO: 48 |
| 8B8 | TYADSVKGRFTISRDNAKNTVYLQMNSLKPGDTAVYYC | SEQ ID NO: 49 |
| 8E10 | | |
| 7G10 | YYADSLKGRFTISRDNAKNTVYLEMNNLKPEDTAVYYC | SEQ ID NO: 50 |
| 7D10 | YYSDSVKGRFTISRDNAKNTVYLQMNSLKPEDTAVYYC | SEQ ID NO: 51 |

TABLE 7
FR4 sequences of 12 single-domain antibodies

| Actual clone number | FR4 | SEQ ID |
|---|---|---|
| 2H7 | WGQGTQVTVSS | SEQ ID NO: 52 |
| 4C5 | | |
| 4D11 | | |
| 5A3 | | |
| 5D1 | | |
| 5D3 | | |
| 7A3 | | |
| 7D10 | | |
| 7G10 | | |
| 7G5 | | |
| 8B8 | | |
| 8E10 | | |

The 12 anti-TfR1 single-domain antibodies have amino acid sequences as set forth in SEQ ID NOS: 1-12, respectively, as shown in Table 8.

TABLE 8
Amino acid sequences of single domain antibodies

| | | Clone |
|---|---|---|
| SEQ ID | Amino acid sequence | number |
| SEQ ID | ESGGGLVQPGESLRLSCVASGSIWSIYTMGWYRQAPGKQRDFVASITG | 2H7 |
| NO: 1 | DSTRTLESVKDRFIISRDNTKNTLYLQMNSLKPEDTAVYYCQAITSLGE | |
| | YWGQGTQVTVSS | |
| SEQ ID | ESGGGLVQPGESLRLSCAASGSIWSIYTMGWYRQAPGLQRDFVGSIAR | 4C5 |
| NO: 2 | EGGRRYAESVKDRFTISRDNAKNTMYLQMNSLKREDTAVYYCNAITS | |
| | LGEYWGQGTQVTVSS | |
| SEQ ID | ESGGGLVQPGESLRLSCVASGSIWSIYTMGWYRQAPGKQRDFVASITR | 4D11 |
| NO: 3 | DSTRTLESVKDRFIISRDNAKNTLYLQMNSLKPEDTAVYYCHAITSLGE | |
| | YWGQGTQVTVSS | |
| SEQ ID | ESGGGLVQAGGSLTLSCAVSRRAFNTKAMGWFRQAPGKEREFVATIS | 5A3 |
| NO: 4 | WSGFSTNYADSVKGRFTISRDNAKNTVYLQMNSLKPGDTAVYYCAA | |
| | GVLDGTNWHEYDYWGQGTQVTVSS | |
| SEQ ID | ESGGGLVQPGESLRLSCVASGSIWSIYTMGWYRQAPGKQRDFVASITR | 5D1 |
| NO: 5 | DGGRRHAESVKDRFTISRDNAKNTLYLQMNSLKPEDTAVYSCLAITSL | |
| | GEYWGQGTQVTVSS | |
| SEQ ID | ESGGGLVEPGGSLRLSCAASGNIFSINNMYWHRQAPGKQRELVGGITR | 5D3 |
| NO: 6 | GGITNYVDSVKGRFTISRDNAKNTVYLQMNSLKPEDTAVYFCNAHPW | |
| | RLGIADYWGQGTQVTVSS | |
| SEQ ID | ESGGGLVQPGESLRLSCVASGSIWSIYTMGWYRQAPGKQRDFVASITR | 7A3 |
| NO: 7 | DSGRRYAESVKDRFTISRDNAKNTLYLQTNSLKPEDTAVYYCHAITSL | |
| | GEYWGQGTQVTVSS | |
| SEQ ID | ESGGGLVQPGGSLRLSCATSGGTFEMYAMAWFRQAPGKDREGVAGFS | 7D10 |
| NO: 8 | WRDSSTTYYSDSVKGRFTISRDNAKNTVYLQMNSLKPEDTAVYYCAA | |
| | RPYVSRWSESSGYDYWGQGTQVTVSS | |
| SEQ ID | ESGGGLVQAGASLKLSCNGSGRTFISYAMGWFRQAPGKQREFVAAER | 7G10 |
| NO: 9 | WSGSGIYYADSLKGRFTISRDNAKNTVYLEMNNLKPEDTAVYYCAAR | |
| | EGGLLHVATSYDYWGQGTQVTVSS | |
| SEQ ID | ESGGGSVQPGGSLRLSCAVSGSIFSIDTMAWYRQAPGKQRELVAAITK | 7G5 |
| NO: 10 | GGSRNYADSVKGRFTISRDNAKNTVYLQMNSLKPEDTAVYYCLADRL | |
| | FRDRSWGQGTQVTVSS | |
| SEQ ID | ESGGGLVQPGGSLRLSCVASGRTFSTSGMGWFRQAPGKEREFVATISW | 8B8 |
| NO: 11 | SGFTTTYADSVKGRFTISRDNAKNTVYLQMNSLKPGDTAVYYCAAGV | |
| | LDSTSWHEYDYWGQGTQVTVSS | |
| SEQ ID | ESGGGLVQAGGSLRLSCAFSGPPFSTYGMGWFRQAPGKEREFVATISG | 8E10 |
| NO: 12 | SGFTTTYADSVKGRFTISRDNAKNTVYLQMNSLKPGDTAVYYCAAGV | |
| | LDGTNWHEYDYWGQGTQVTVSS | |

The nucleic acid molecules encoding the 12 anti-TfR1 single-domain antibodies have the nucleotide sequences as set forth in SEQ ID NOS: 13-24, respectively, as shown in Table 9.

TABLE 9
Nucleotide sequences of the nucleic acid encoding the single domain antibodies

| | | Clone |
|---|---|---|
| SEQ ID | Nucleotide sequence | number |
| SEQ ID | GAGTCTGGGGGAGGCTTGGTGCAGCCTGGGGAGTCTCTGAGACTCTCCT | 2H7 |
| NO: 13 | GTGTAGCCTCTGGAAGCATCTGGAGTATCTATACCATGGGCTGGTACCGC | |
| | CAGGCTCCAGGGAAGCAGCGCGACTTCGTCGCAAGTATTACAGGTGATA | |
| | GTACTAGGACGTTAGAGTCCGTGAAGGACCGATTTATTATCTCCAGAGAC | |
| | AACACCAAGAACACGTTGTATCTGCAAATGAACAGTCTGAAACCTGAGG | |
| | ACACGGCCGTCTATTACTGTCAGGCAATCACTTCGCTTGGAGAATACTGG | |
| | GGCCAGGGGACCCAGGTCACCGTCTCCTCA | |
| SEQ ID | GAGTCTGGGGGAGGCTTGGTGCAGCCTGGGGAGTCTCTGAGACTCTCCT | 4C5 |
| NO: 14 | GTGCAGCCTCTGGAAGCATCTGGAGTATCTATACCATGGGCTGGTACCGC | |
| | CAGGCTCCAGGACTGCAGCGCGACTTCGTCGGAAGTATAGCGCGTGAAG | |
| | GTGGTAGGAGATATGCAGAGTCCGTGAAGGACCGATTCACCATCTCCAG | |
| | AGACAACGCCAAGAACACGATGTATCTGCAAATGAACAGTCTGAAACGT | |
| | GAAGACACGGCCGTCTATTACTGTAATGCAATCACTTCGCTTGGAGAATA | |
| | CTGGGGCCAGGGGACCCAGGTCACCGTCTCCTCA | |
| SEQ ID | GAGTCTGGAGGAGGCTTGGTGCAGCCTGGGGAGTCTCTGAGACTCTCCT | 4D11 |
| NO: 15 | GTGTAGCCTCTGGAAGCATCTGGAGTATCTATACCATGGGCTGGTACCGC | |
| | CAGGCTCCAGGGAAGCAGCGCGACTTCGTCGCAAGTATTACGCGTGATA | |
| | GTACTAGGACGTTAGAGTCCGTGAAGGACCGATTTATTATCTCCAGAGAC | |
| | AACGCCAAGAACACGTTGTATCTGCAAATGAACAGTCTGAAACCTGAGG | |
| | ACACGGCCGTCTATTACTGTCATGCAATCACTTCGCTTGGAGAATACTGG | |
| | GGCCAGGGGACCCAGGTCACCGTCTCCTCA | |
| SEQ ID | GAGTCTGGGGGAGGATTGGTGCAGGCTGGGGGCTCTCTGACACTCTCCT | 5A3 |
| NO: 16 | GTGCAGTCTCTCGACGCGCCTTCAATACCAAAGCTATGGGCTGGTTCCGC | |
| | CAGGCTCCAGGGAAGGAGCGTGAATTTGTAGCAACTATTAGTTGGAGTG | |
| | GTTTTAGCACAAACTATGCAGACTCCGTGAAGGGCCGATTCACCATCTCC | |
| | AGAGACAACGCCAAGAACACGGTGTATCTGCAAATGAACAGCCTGAAA | |
| | CCTGGGGACACGGCCGTTTATTACTGTGCAGCGGGGGTCTTAGACGGTAC | |
| | TAATTGGCATGAATATGACTACTGGGGCCAGGGGACCCAGGTCACCGTCT | |
| | CCTCA | |
| SEQ ID | GAGTCTGGAGGAGGCTTGGTGCAGCCTGGGGAGTCTCTGAGACTCTCCT | 5D1 |
| NO: 17 | GTGTAGCCTCTGGAAGCATCTGGAGTATCTATACCATGGGCTGGTACCGC | |
| | CAGGCTCCAGGGAAGCAGCGCGACTTCGTCGCAAGTATTACGCGTGATG | |
| | GTGGTAGGCGCCATGCAGAGTCCGTGAAGGACCGATTCACCATCTCCAG | |
| | AGACAACGCCAAGAACACGTTGTATCTGCAAATGAACAGTCTGAAACCT | |
| | GAGGACACGGCCGTCTATTCGTGCCTTGCAATCACTTCGCTTGGAGAATA | |
| | CTGGGGCCAGGGGACCCAGGTCACCGTCTCCTCA | |
| SEQ ID | GAGTCTGGGGGAGGCTTGGTGGAGCCTGGGGGGTCTCTGAGACTCTCCT | 5D3 |
| NO: 18 | GTGCAGCCTCTGGAAACATCTTCAGTATCAATAACATGTACTGGCACCGC | |
| | CAGGCTCCAGGGAAGCAGCGCGAGCTGGTCGGAGGTATTACTAGAGGTG | |
| | GTATCACAAACTATGTAGACTCCGTGAAGGGCCGATTCACCATCTCCAGA | |
| | GACAACGCCAAGAACACGGTGTATCTGCAAATGAACAGCCTGAAACCTG | |
| | AGGACACGGCCGTCTATTTCTGTAATGCCCACCCGTGGCGGCTTGGGATT | |
| | GCTGACTACTGGGGCCAGGGGACCCAGGTCACCGTCTCCTCA | |
| SEQ ID | GAGTCTGGAGGAGGCTTGGTGCAGCCTGGGGAGTCTCTGAGACTCTCCT | 7A3 |
| NO: 19 | GTGTAGCCTCTGGAAGCATCTGGAGTATCTATACCATGGGCTGGTACCGC | |
| | CAGGCTCCAGGGAAGCAGCGCGACTTCGTCGCAAGTATTACGCGTGATA | |
| | GTGGTAGGAGGTATGCAGAGTCCGTGAAGGACCGATTCACCATCTCCAG | |
| | AGACAACGCCAAGAACACGTTGTATCTGCAAACGAACAGTCTGAAACCT | |
| | GAGGACACGGCCGTCTATTACTGTCATGCAATCACTTCGCTTGGAGAATA | |
| | CTGGGGCCAGGGGACCCAGGTCACCGTCTCCTCA | |
| SEQ ID | GAGTCTGGAGGAGGCTTGGTGCAGCCTGGGGGGTCTCTGAGACTCTCCT | 7D10 |
| NO: 20 | GTGCAACCTCTGGTGGCACCTTCGAAATGTATGCCATGGCGTGGTTCCGC | |
| | CAGGCTCCAGGGAAGGATCGTGAGGGTGTAGCAGGTTTTAGCTGGAGAG | |
| | ATAGTAGTACAACATACTATTCAGACTCCGTGAAGGGCCGATTCACCATCT | |
| | CCAGAGACAACGCCAAGAACACGGTGTATCTGCAAATGAACAGCCTGAA | |
| | ACCTGAAGACACGGCCGTTTATTACTGTGCAGCCCGACCGTACGTTAGTA | |
| | GGTGGTCGGAATCAAGCGGGTATGACTACTGGGGCCAGGGGACCCAGGT | |
| | CACCGTCTCCTCA | |
| SEQ ID | GAGTCTGGAGGAGGATTGGTGCAGGCTGGGGCCTCTCTGAAACTCTCCT | 7G10 |
| NO: 21 | GTAATGGTTCTGGACGCACCTTCATTAGCTATGCCATGGGCTGGTTCCGCC | |
| | AGGCTCCAGGGAAGCAGCGTGAGTTTGTTGCAGCTGAGAGGTGGAGTG | |
| | GTAGTGGCATATACTATGCAGACTCCTTGAAGGGCCGATTCACCATCTCCA | |
| | GAGACAACGCCAAGAACACGGTGTATCTGGAAATGAACAACCTGAAAC | |
| | CTGAGGACACGGCCGTTTATTACTGTGCAGCACGTGAGGGAGGATTACT | |
| | GCATGTAGCGACTTCGTATGACTACTGGGGCCAGGGGACCCAGGTCACC | |
| | GTCTCCTCA | |
| SEQ ID | GAGTCTGGAGGAGGCTCGGTGCAGCCTGGGGGGTCTCTGAGACTCTCCT | 7G5 |
| NO: 22 | GTGCAGTCTCTGGAAGCATCTTCAGTATCGATACCATGGCCTGGTACCGC | |
| | CAGGCTCCAGGGAAGCAGCGCGAGTTGGTCGCAGCTATTACTAAGGGTG | |
| | GTAGTAGAAACTATGCAGACTCCGTGAAGGGCCGATTCACCATCTCCAGA | |
| | GACAACGCCAAGAACACGGTGTATCTACAAATGAACAGCCTGAAACCTG | |
| | AGGACACAGCCGTCTATTACTGTCTAGCAGATAGACTCTTCCGAGATCGT | |
| | TCCTGGGGCCAGGGGACCCAGGTCACCGTCTCCTCA | |
| SEQ ID | GAGTCTGGAGGAGGATTGGTGCAGCCTGGGGGGTCTCTGAGACTTTCCT | 8B8 |
| NO: 23 | GTGTAGCCTCTGGACGCACCTTCAGTACCTCCGGCATGGGCTGGTTCCGC | |
| | CAGGCTCCAGGGAAGGAGCGTGAATTTGTAGCAACTATTAGTTGGAGTG | |
| | GTTTTACGACAACCTATGCAGACTCCGTGAAGGGCCGATTCACCATCTCC | |
| | AGAGACAACGCCAAGAACACGGTGTATCTGCAAATGAACAGCCTGAAA | |
| | CCTGGGGACACGGCCGTTTATTACTGTGCAGCGGGGGTCTTAGACAGTAC | |
| | TAGTTGGCATGAATATGACTACTGGGGCCAGGGGACCCAGGTCACCGTCT | |
| | CCTCA | |
| SEQ ID | GAGTCTGGAGGAGGATTGGTGCAGGCTGGGGGCTCTCTGAGACTCTCCT | 8E10 |
| NO: 24 | GTGCATTCTCTGGACCCCCCTTCAGTACCTATGGCATGGGCTGGTTCCGC | |
| | CAGGCTCCAGGGAAGGAGCGTGAATTTGTAGCAACTATTAGTGGGAGTG | |
| | GTTTTACAACAACCTATGCAGACTCCGTGAAGGGCCGATTCACCATCTCC | |
| | AGAGACAACGCCAAGAACACGGTGTATCTGCAAATGAACAGCCTGAAA | |
| | CCTGGGGACACGGCCGTTTATTACTGTGCAGCGGGGGTCTTAGATGGTAC | |
| | TAATTGGCATGAATATGACTACTGGGGCCAGGGGACCCAGGTCACCGTCT | |
| | CCTCA | |

### Example 5: Purification and Expression of Anti-Humanized TfR1 Protein Single-Domain Antibodies in E. coli

The plasmids from the different clones obtained in Example 4 (i.e., pMECS vectors containing VHH) were electroporated into E. coli HB2151 (purchased from Agilent) and plated onto LB medium plate containing ampicillin and glucose, followed by overnight incubation at 37° C. Single colonies were selected and inoculated into 5 mL of LB broth (purchased from Sangon Biotech, Cat. No. A507002-0250) containing ampicillin, followed by overnight incubation with shaking at 37° C.

1 mL of the overnight incubated bacterial solution was inoculated into 330 mL of TB broth, a mixture of TB (A) and TB (B). TB (A) included tryptone (purchased from OXOID, Cat. No. 73049-73-7), yeast extract (purchased from OXOID, Cat. No. 8013-01-2), and glycerol (purchased from Sangon Biotech, Cat. No. A501745). TB (B) included potassium dihydrogen phosphate (purchased from Sangon Biotech, Cat. No. A600445) and dipotassium hydrogen phosphate (purchased from Sangon Biotech, Cat. No. A501212). The culture was incubated in a shaker at 37° C. until the OD at 600 nm reached 0.6-0.9. Subsequently, 1 M IPTG was added, and the culture was incubated in a shaker overnight at 28° C. E. coli cells were harvested by centrifugation. Crude antibody extracts were obtained using osmotic lysis. Antibodies were purified via nickel column affinity chromatography.

### Example 6: Construction of Recombinant Eukaryotic Expression Vectors for Anti-Humanized TfR1 Single-Domain Antibody-Fc Fusion Antibodies

The nucleotide sequences of the 12 single-domain antibodies (SEQ ID NOS: 13-24) and other single-domain antibodies having sequences unlisted obtained in Example 4 were individually incorporated into an eukaryotic expression vector RJK-V4-hFC1 designed and modified by Nanjing Jietai Biotechnology Co., Ltd. to obtain recombinant eukaryotic expression vectors. The modification method for the eukaryotic expression vector RJK-V4-hFC will be described in Example 10. The resulting recombinant eukaryotic expression vectors were transformed into E. coli DH5α, cultured, and subjected to plasmid extraction with endotoxin removal. The extracted plasmids were sequenced to confirm the correct recombinant eukaryotic expression vectors, which were subsequently used for transfection into eukaryotic cells. The VHH-Fc fusion antibodies were expressed using the methods described in Example 7 or 8 and purified according to the protocol in Example 9.

### Example 7: Expression of Anti-Humanized TfR1 Protein Single-Domain Antibodies in ExpiCHO-S Cells

Three days prior to transfection, ExpiCHO-S™ cells (from Thermo Fisher) were passaged at a cell concentration of 2.5×10⁵ cells/mL and expanded. A required volume of cell culture was transferred to a 500 mL shake-flask containing 120 mL (final volume) of fresh, pre-warmed ExpiCHO™ Expression Medium to achieve a cell concentration of 4×10⁶ to 6×10⁶ viable cells/mL. One day before transfection, ExpiCHO-S™ cells were diluted to a concentration of 3.5×10⁶ viable cells/mL and incubated overnight.

On the day of transfection, cell density and viability were measured. The cell density reached approximately 7×10⁶ to 10×10⁶ viable cells/mL.

The cells were diluted to 6×10⁶ viable cells/mL using fresh ExpiCHO™ Expression Medium pre-warmed to 37° C. A required volume of cells were then transferred to a 500 mL shake-flask containing 100 mL (final volume) of fresh, pre-warmed ExpiCHO™ Expression Medium. ExpiFectamine™ CHO Reagent (from Thermo Fisher) was gently inverted and mixed. ExpiFectamine™ CHO Reagent was diluted with 3.7 mL OptiPRO™ Medium (Thermo Fisher), and shaked or mixed well. The recombinant eukaryotic expression vectors expressing VHH-Fc fusion antibodies prepared in Example 6 were diluted into 4 mL OptiPRO™ Medium, and mixed well by shaking.

The diluted ExpiFectamine™ CHO Reagent and the diluted recombinant eukaryotic expression vectors were incubated at room temperature for 1-5 minutes, then gently added to the prepared ExpiCHO-S™ cell suspension with gently shaking the shake-flask. The cell suspension was incubated in a 37° C., 8% CO₂ incubator with shaking.

On Day 1 post-transfection (after about 18-22 hours), 600 μL ExpiFectamine™ CHO Enhancer and 24 mL ExpiCHO Feed were added. The supernatants of the cell culture were harvested on Day 8 post-transfection when cell viability dropped below 70%.

### Example 8: Expression of Anti-Humanized TfR1 Protein Single-Domain Antibodies in 293F Cells

Three days prior to transfection, 293F cells were passaged at a cell concentration of 2.5×10⁵ cells/mL and expanded. A required volume of cell culture was transferred to a 500 mL shake-flask containing 120 mL (final volume) of fresh, pre-warmed OPM-293 CD05 Medium (purchased from OPM Biosciences (Suzhou) Co., LTD, Cat. No. 81075-001) to achieve a cell concentration of 2×10⁶ to 3×10⁶ viable cells/mL. On the day of transfection, cell density and viable cell percentage were measured. The cells reaching at a cell density of approximately 2×10⁶ to 3×10⁶ viable cells/mL were used for transfection.

The cells were diluted to 1×10⁶ viable cells/mL using pre-warmed OPM-293 CD05 Medium. A required volume of cells was then transferred to a 500 mL shake-flask containing 100 mL (final volume) of fresh, pre-warmed OPM-293 CD05 Medium.

4 mL Polyethylenimide reagent (PEI, 1 mg/mL, purchased from PolyScience Inc., Cat. No. A795153) was diluted with 4 mL Opti-MEM medium, and mixed well by shaking or pipetting. The recombinant eukaryotic expression vectors expressing VHH-Fc fusion antibodies prepared in Example 6 were diluted into 4 mL Opt-MEM medium, and mixed well by shaking, filtered through a 0.22 μm filter, and incubated at room temperature for 5 minutes.

The diluted PEI reagent and the diluted recombinant eukaryotic expression vectors were incubated at room temperature for 15-20 minutes, then gently added to the prepared 293F cell suspension with gently shaking the shake-flask. The cell suspension was incubated in a 37° C., 5% CO₂ incubator with shaking at 120 rpm. 5 mL of OPM-CHO PFF05 feed was added at 24 and 72 hours post-transfection. The supernatants of the cell culture were harvested on Day 7 post-transfection when cell viability dropped below 70%.

### Example 9: Purification of Anti-Humanized TfR1 Protein Single-Domain Antibodies

The supernatants obtained from Example 7 and Example 8 were filtered respectively through 0.45 μm disposable filters to remove insoluble impurities.

The filtrate was purified by affinity chromatography using a protein purification instrument. Specifically, the antibodies were captured by Protein A-coupled agarose filler (purchased from Suzhou Nanomicro Technology Co., Ltd., Cat. No. 17010-250100-2100), leveraging the binding affinity between Protein A and the humanized Fc region.

The filtrate was loaded onto a Protein A pre-packed column at a flow rate of 1 mL/min, allowing the antibodies in the filtrate to bind to Protein A. Low-salt buffer and high-salt buffer were sequentially used to wash away impurity proteins.

Antibodies were eluted using a low-pH elution buffer.

The eluate containing antibodies was immediately neutralized by adding pH 9.0 Tris-HCl solution.

The neutralized antibody solution was dialyzed, followed by SDS-PAGE analysis. The purity of the obtained antibodies was higher than 95%. The antibody solutions with concentrations higher than 0.5 mg/mL were stored at low temperature for subsequent use.

### Example 10: Modification of Eukaryotic Expression Vector for Single-Domain Antibody

The eukaryotic expression vector RJK-V4-hFc1 for single-domain antibody was prepared by fusing the coding sequence of the human IgG1 Fc domain to the vector pcDNA™3.4 (obtained from Invitrogen). The vector RJK-V4-hFC1 contains the heavy chain hinge region (Hinge), CH2 region, and CH3 region of IgG1.

The modification protocol was as follows:

The restriction enzyme sites XbaI and AgeI on the vector pcDNA3.4 were selected;

A multiple cloning site and a 6×His tag were introduced via overlap PCR into the 5′ end and 3′ end of the coding sequence of the human IgG1 Fc region, respectively;

A pair of primers containing the XbaI and AgeI restriction enzyme sites respectively were used to amplify the aforementioned sequence by PCR;

The pcDNA3.4 and the amplified sequence obtained above were digested with the restriction enzymes XbaI and AgeI, respectively;

The digested vector and digested amplified sequence were ligated using T4 ligase, and the ligation product was transformed into E. coli for amplification and sequencing verification, thereby obtaining the expression vector containing the coding sequence of the Fc region.

### Example 11: Expression and Purification of Tool Antibodies (Tab) Targeting Humanized TfR1 Protein

Herein, Tab1 is the antibody AB405 targeting humanized TfR1, with sequences derived from WO2014089209A2. Tab2 is the antibody RG6102 targeting humanized TfR1, with sequences derived from WO2017055540A1. Tab4 is pabinafusp alfa targeting humanized TfR1 (see, U.S. Pat. No. 11,111,308B2). Tab1, Tab2, and Tab4 were used as positive control antibodies for the anti-TfR1 single-domain antibodies in the present disclosure. The retrieved sequences were commissioned to General Biosystems (Anhui) Co., Ltd. for mammalian cell expression system codon optimization, cloned into the pcDNA3.1 vector, and transformed into E. coli. Following antibiotic resistance screening, positive clones were selected for amplification. Plasmids were then extracted using a Midiprep Kit (Macherey Nagel, Cat #740412.50). Polyethylenimine (PEI) dilution solution and a solution containing plasmid expressing repective Tab1, Tab2, or Tab4 at 100 μg plasmid (40 μg heavy chain+60 μg light chain) per 100 mL of cells were added to the culture medium of 293F cells (medium: FreeStyle 293 Expression medium, Thermo Fisher, Cat #12338026, supplemented with F-68, Thermo Fisher, Cat #24040032), respectively, for transient expression. At 6-24 hours post-transfection, 5 vol. % of 10% Peptone (Sigma, Cat #P0521-100G) was added, and the cells were cultured under 8% CO₂ at 130 rpm for approximately 7-8 days. Supernatants were collected when cell viability dropped to 50%. The antibodies were purified using Protein A (GE, Cat #17-5438-02) gravity columns. The purified antibodies were dialyzed against PBS, measured for their concentrations by Nanodrop, assessed for their purity via SEC, and validated for their binding activity by indirect ELISA.

The Tab1, Tab2, Tab3, and Tab4 obtained through this method exhibited a concentration of >2 mg/mL and purity of >95%.

### Example 12: Determination of Dose-Response Curves for Antibody-Antigen Binding

This example was performed using a standard enzyme-linked immunosorbent assay (ELISA).
(1) The wells of an ELISA plate were coated with 50 μL of 1 μg/mL TfR1 protein at 4° C. overnight.
(2) The wells of the ELISA plate were washed multiple times with PBST buffer. After adding 200 μL of 5% milk, the plate was blocked at 37° C. for 2 h.
(3) The anti-humanized TfR1 single-domain antibody-Fc fusion antibodies (i.e., VHH-hFc fusion antibodies expressed in 293F cells) prepared in Example 9 were diluted to 2 μg/mL, followed by 5-fold serial dilutions to generate 8 concentration gradients. Additionally, control wells for hIgG, Tab1, Tab2, and Tab4 were set up. Tab1, Tab2, and Tab4 were prepared in Example 11. hIgG referred to an isotype control that did not bind to any TfR1 protein and was commercially available.
(4) The wells of the ELISA plate from (2) were washed multiple times with PBST buffer. After adding 50 μL of the diluted single-domain antibodies, hIgG, Tab1, Tab2, or Tab4 from step (3) to respective wells in duplicate, the plate was incubated at 37° C. for 1 hour.
(5) The wells of the ELISA plate from (4) were washed multiple times with PBST buffer. After adding 50 μL of HRP-Goat anti-hIgG secondary antibody to each well, the plate was incubated at 37° C. for 30 min.
(6) The wells of the ELISA plate from (5) were washed multiple times with PBST buffer. After adding 50 μL of TMB chromogenic solution to each well, the reaction was carried out in the dark at room temperature for 15 min.
(7) 50 LL of stop solution (1 mol/L HCl) was added to each well to terminate the reaction. OD450 was measured using a microplate reader, and the readings were recorded.
(8) The curves were plotted to calculate EC50 values.

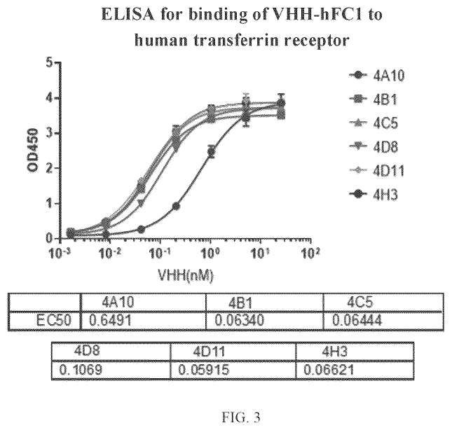

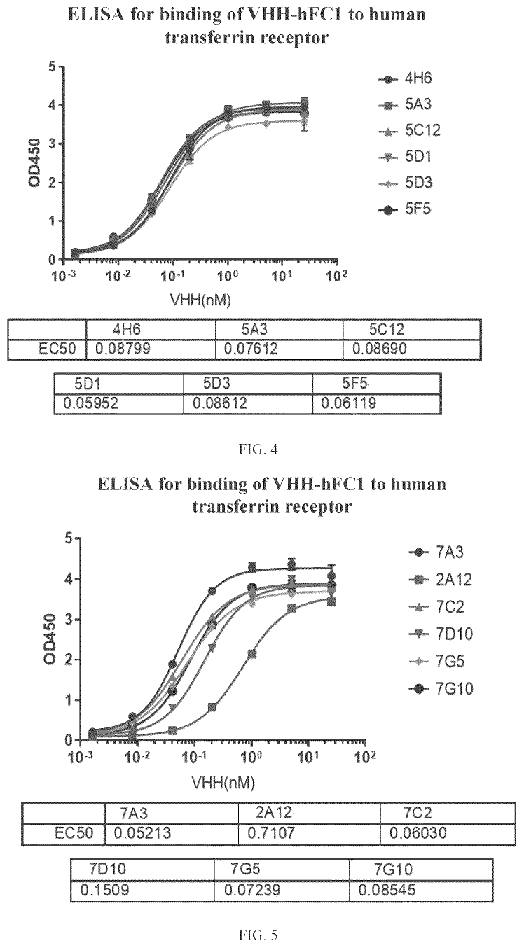

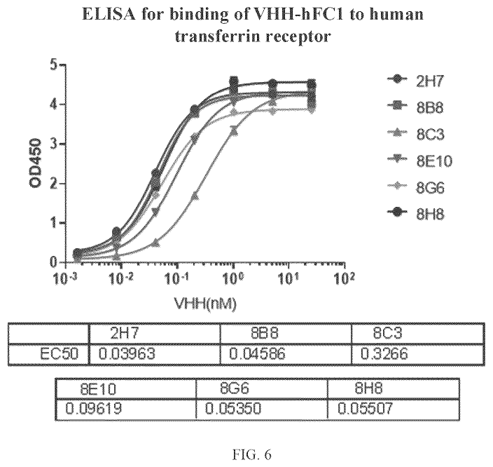

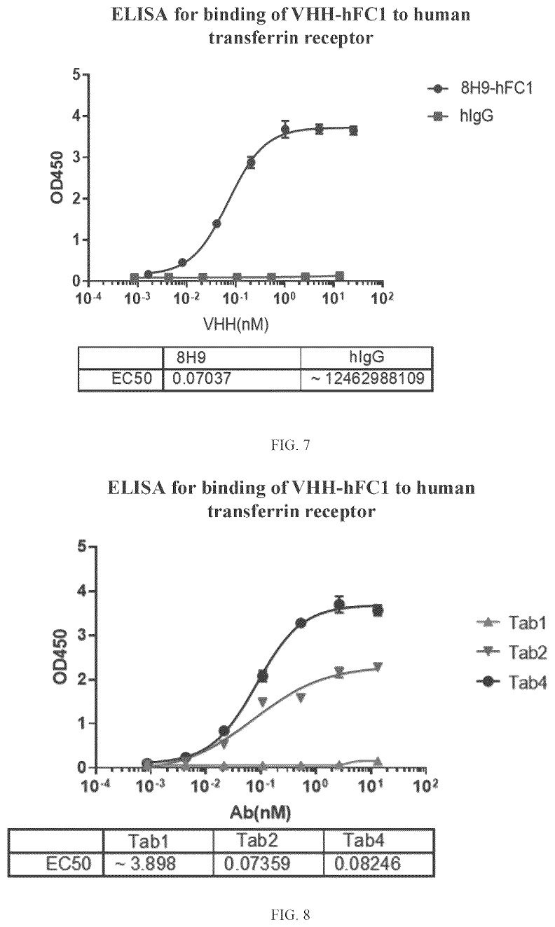

The experimental results are shown in FIGS. 3 to 8. According to FIGS. 3 to 8, the EC50 values of the antibodies of the present disclosure (5D3, 7G10, 8E10, 5A3, 4C5, 2H7, 4D11, 5D1, 7A3, 7D10, 8D8, 7G5) were lower than or comparable to those of Tab, demonstrating their excellent antigen-binding activity.

### Example 13: Internalization of Human Erythroleukemia Cells (TF-1 Cells)

### I. Preparation of PHrodo Conjugates

1) 100 μg of the purified anti-humanized TfR1 single-domain antibody-Fc fusion antibodies (i.e., VHH-hFc fusion antibodies expressed in 293F cells) prepared in Example 9, 100 μg of Tabs prepared in Example 11, and 100 μg of hIgGTfR1 were labeled with pHrodo™ iFL Green STP Ester (Thermo Fisher, Cat. No. P36013) to obtain solutions of pHrodo-VHH-hFcs, pHrodo-Tabs, or pHrodo-hIgG.
2) The solutions of pHrodo-RS40-VHH-hFcs, pHrodo-Tabs, or pHrodo-hIgG were desalted.
3) The protein concentrations in the solutions were measured using Pierce™ BCA Protein Assay Kit (from Thermo Fisher, Cat. No. 23227).

### II. Internalization Assay

1) TF-1 cells (Procell CL-0232, from Wuhan Pricella Biotechnology Co., Ltd.) were harvested by trypsinization and centrifugation.
2) 1×10⁶ TF-1 cells were washed once with cold PBS. 10 μg/mL pHrodo-Tabs, 10 μg/mL pHrodo-hIgG, or 10 μg/mL pHrodo-VHH-hFcs was added to the cold PBS.
3) The cell suspension from step (2) was divided into two aliquots. One aliquot of the cell suspension was incubated on ice, and the other was incubated at 37° C. for 4 hours.
4) The cells after incubation in step (3) were washed once with cold PBS and resuspended in 200 μL cold PBS.
5) FITC signals were detected using a CytoFlex flow cytometer (from Beckman Coulter).

The internalization assay results for the 12 anti-humanized TfR1 single-domain antibody-Fc fusion antibodies, Tab4 (positive control), and hIgG (negative control) are shown in Table 10. In the table, “Fold” represents the ratio of MFI at 37° C. to MFI at 4° C. The MFI at 4° C. represents the background value. This example demonstrates that the anti-humanized TfR1 single-domain antibodies can be internalized by target cells so as to exert pharmacological effects in the cells.

TABLE 10
Results of TF-1 cell internalization assay
TF-1 internalization by binding ELISA

| Clone number | 37° C. MFI | 4° C. MFI | fold |
|---|---|---|---|
| 4C5-hFC1 | 1842 | 759 | 2.42687747 |
| 4D11-hFC1 | 1327 | 713 | 1.86115007 |
| 5A3-hFC1 | 1666 | 791 | 2.10619469 |
| 5D1-hFC1 | 1402 | 751 | 1.866844208 |
| 5D3-hFC1 | 545 | 576 | 0.946180556 |
| 7A3-hFC1 | 1865 | 762 | 2.447506562 |
| 7D10-hFC1 | 2343 | 954 | 2.455974843 |
| 7G5-hFC1 | 523 | 482 | 1.085062241 |
| 7G10-hFC1 | 1535 | 731 | 2.099863201 |
| 2H7-hFC1 | 1448 | 744 | 1.946236559 |
| 8B8-hFC1 | 1279 | 687 | 1.861717613 |
| 8E10-hFC1 | 1123 | 678 | 1.656342183 |
| Tab4 | 1304 | 712 | 1.831460674 |
| hIgG | 476 | 471 | 1.010615711 |

### Example 14: Internalization of CHO-K1-TfR1 Cells

### I. Preparation of PHrodo Conjugates

1) 100 μg of the purified anti-humanized TfR1 single-domain antibody-Fc fusion antibodies (i.e., VHH-hFc fusion antibodies expressed in 293F cells) prepared in Example 9, 100 μg of Tabs prepared in Example 11, and 100 μg of hIgGTfR1 were labeled with pHrodo™ iFL Green STP Ester (Thermo Fisher, Cat. No. P36013) to obtain solutions of pHrodo-VHH-hFcs, pHrodo-Tabs, or pHrodo-hIgG.
2) The solutions of pHrodo-VHH-hFcs, pHrodo-Tabs, or pHrodo-hIgG were desalted.
3) The protein concentrations in the solutions were measured using Pierce™ BCA Protein Assay Kit (Thermo Fisher, Cat. No. 23227).

### II. Internalization Assay

1) CHO-K1-TfR1 cells were harvested by trypsinization and centrifugation. CHO-K1-TfR1 cells were derived from the CHO-K1-TfR1 cell line, and the CHO-K1-TfR1 cell line was established through the following steps. The coding sequence (CDS) region information of TfR1 (accession number: NM_003234.4) was obtained from the NCBI database. The DNA sequence of the CDS region was synthesized by gene synthesis, and cloned into the PHB-Puro expression vector to construct PHB-TfR1-Puro plasmid vector. Lentivirus was subsequently packaged and used to infect CHO-K1 cells to introduce the CMV-TfR1-EF1a-Puromycin gene into the CHO-K1 genome. Puromycin resistance screening and monoclonal screening were performed to obtain the CHO-K1-TfR1 stable cell line stably expressing the TfR1 protein.
2) 1×10⁶ CHO-K1-TfR1 cells were washed once with cold PBS. 10 μg/ml pHrodo-RS40-Tabs, 10 μg/mL pHrodo-hIgG, or 10 μg/mL pHrodo-RS40-VHH-hFcs was added to the cold PBS.
3) The cell suspension from step (2) was divided into two aliquots. One aliquot of the cell suspension was incubated on ice, and the other was incubated at 37° C. for 4 hours.
4) The cells after incubation in step (3) were washed once with cold PBS and resuspended in 200 μL cold PBS.
5) FITC signals were detected using a CytoFlex flow cytometer (from Beckman Coulter).

The internalization assay results for the 12 anti-humanized TfR1 single-domain antibody-Fc fusion antibodies, Tab4 (positive control), and hIgG (negative control) are shown in Table 11. In the table, “Fold” represents the ratio of MFI at 37° C. to MFI at 4° C. The MFI at 4° C. represents the background value. This example demonstrates that the anti-humanized TfR1 single-domain antibodies can be internalized by target cells so as to exert pharmacological effects in the cells.

TABLE 11
Results of CHO-K1-TfR1 internalization assay
CHO-K1-TfR1 internalization by binding ELISA

| Clone number | 37° C. MFI | 4° C. MFI | fold |
|---|---|---|---|
| 4C5-hFC1 | 1129 | 481 | 2.347193347 |
| 4D11-hFC1 | 1162 | 497 | 2.338028169 |
| 5A3-hFC1 | 1264 | 491 | 2.574338086 |
| 5D1-hFC1 | 1323 | 502 | 2.635458167 |
| 5D3-hFC1 | 943 | 451 | 2.090909091 |
| 7A3-hFC1 | 1697 | 536 | 3.166044776 |
| 7D10-hFC1 | 2130 | 625 | 3.408 |
| 7G5-hFC1 | 666 | 457 | 1.457330416 |
| 7G10-hFC1 | 1189 | 503 | 2.363817097 |
| 2H7-hFC1 | 1358 | 526 | 2.581749049 |
| 8B8-hFC1 | 1356 | 512 | 2.6484375 |
| 8E10-hFC1 | 1037 | 488 | 2.125 |
| Tab4 | 1226 | 520 | 2.357692308 |
| hIgG | 711 | 464 | 1.532327586 |

### Example 15: Epitope Experiment

### I. Epitope Peptides of TfR1 Protein

After extensive theoretical research and experimental exploration, the present inventors performed protein three-dimensional structure preprocessing through computer-assisted protein structure analysis, and docked the processed antigen and antibody (2H7, or 5D1) structures with the output 1000 conformations, selected the docking structure with the highest score as the final result, and determined the binding region of the antigen and antibody in these results as the epitope peptide region.

TABLE 12
Peptide names and their amino acid sequences

| | | | Number |
|---|---|---|---|
| | | | of amino |
| No. | Peptide name | Amino acid sequence | acids |
| 1 | D1-helix1 | NLDYERYNSQLLSFVRDLNQYRADIKEM (SEQ ID NO: 83) | 28 |
| 2 | D1-helix2 | GLSLQWLYSARGDFFRATSRLTTDFGNAEK (SEQ ID NO: 84) | 30 |
| 3 | D1-helix4 | SPKESPFRHVFWGSGSHTLPALLENLKLRKQNNGAF (SEQ ID | 36 |
| | | NO: 85) | |
| 4 | D1-helix2- | RATSRLTTDFGNAEKTDRFVMKKLNDRV (SEQ ID NO: 86) | 28 |
| | helix3 | | |
| 5 | D3-helix1 | RLYWDDLKRKLSEKLDSTD (SEQ ID NO: 87) | 19 |
| 6 | D3-helix2 | FTSTIKLLNENSYV (SEQ ID NO: 88) | 14 |
| 7 | D3-helix3 | EAGSQKDENLALYVENQFREFK (SEQ ID NO: 89) | 22 |
| 8 | D3-helix1- | RLYWDDLKRKLSEKLDSTDFTSTIKLLNENSYV (SEQ ID | 33 |
| | helix2 | NO: 90) | |
| 9 | D3-helix2- | FTSTIKLLNENSYVPREAGSQKDENLALYVENQFREFK (SEQ ID | 38 |
| | helix3 | NO: 91) | |
| 10 | D3-helix3- | EAGSQKDENLALYVENQFREFKLSKVWRDQHFV (SEQ ID | 33 |
| | loop1 | NO: 92) | |
| 11 | D3-loop1 | LSKVWRDQHFV (SEQ ID NO: 93) | 11 |
| 12 | D2-loop1 | GTKKDFEDLY (SEQ ID NO: 94) | 10 |
| 13 | D2-loop1-K | VHANFGTKKDFEDLYTPVNG (SEQ ID NO: 95) | 20 |
| 14 | D2-loop3-K | NMEGDCPSDWKTDSTCRMVT (SEQ ID NO: 96) | 20 |
| 15 | D2-loop1- | GTKKDFEDLYTPVNGSIVIVRAGKITF (SEQ ID NO: 97) | 27 |
| | loop2 | | |
| 16 | D1-helix3 | TDRFVMKKLNDRVMRVEYHFLSPYV (SEQ ID NO: 98) | 25 |
| 17 | D1-helix5 | NETLFRNQLALATWTIQGAANALSGDVWDIDN (SEQ ID | 32 |
| | | NO: 99) | |
| 18 | D1-helix1- | VRDLNQYRADIKEMGLSLQWLYSARGDFF (SEQ ID NO: 100) | 29 |
| | helix2 | | |
| 19 | D1-helix3- | MRVEYHFLSPYVSPKESPFRHVFWGSGSHT (SEQ ID NO: 101) | 30 |
| | helix4 | | |
| 20 | D1-helix4- | LPALLENLKLRKQNNGAFNETLFRNQLALATWTI (SEQ ID | 34 |
| | helix5 | NO: 102) | |
| 21 | D2-loop2-K | SIVIVRAGKITFAEKVANAE (SEQ ID NO: 103) | 20 |

Based on the analysis results, Nanjing Jietai Biotechnology Co., Ltd. was commissioned to synthesize peptides corresponding to the aforementioned amino acid sequences. The binding of peptides to antibodies was assessed by ELISA, with positive controls included to confirm the final antigen-antibody binding epitopes.

### 1.3 Identification of Peptide-Antibody Binding

The peptide-antibody binding was identified using a standard enzyme-linked immunosorbent assay (ELISA). The steps were as follows:
(1) The wells of an ELISA plate were coated with 100 μL of 2 μg/mL SA (streptavidin) protein at 4° C. overnight.
(2) The wells of the ELISA plate were washed with PBST buffer. After adding 200 μL of 2% BSA, the plate was blocked at 37° C. for 1 h.
(3) The peptide protein was diluted to 2 μg/mL, and a negative control and a positive control (AVI-tagged TfR1 protein) were included. Each experiment contained duplicate controls. In the results, the result of positive control was denoted as N.
(4) The wells of the ELISA plate were washed multiple times with PBST buffer. Then, 100 μL of the diluted peptide protein solution from step (3) was added into wells in duplicate, and along with the negative control wells, incubated at 37° C. for 1 h.
(5) The wells of the ELISA plate were washed multiple times with PBST buffer. Next, 100 μL of 2 μg/mL TfR 1 antibody 2H7-hFc solution was added, and the plate was incubated at 37° C. for 1 h. Here, 2H7-hFc refers to the anti-humanized TfR1 single-domain antibody-Fc fusion antibody expressed in 293F cells and purified.
(6) The wells of the ELISA plate were washed multiple times with PBST buffer. Then, 100 μL of 2× anti-hFc HRP secondary antibody was added, and the plate was incubated at 37° C. for 1 h.
(7) The wells of the ELISA plate were washed multiple times with PBST buffer. After adding 100 μL of TMB chromogenic solution, the reaction was carried out in the dark at room temperature for 30 min.
(8) 100 μL of stop solution (1 mol/L HCl) was added to terminate the reaction. OD450 was measured using a microplate reader, and the readings were recorded.

TABLE 13
Peptide names and their assay results

| Polypeptide name | OD450 | N | P/N |
|---|---|---|---|
| D1-helix1 | 0.2105 | 0.171 | 1.2 |
| D1-helix2 | 0.2595 | 0.135 | 1.9 |
| D1-helix4 | 1.4935 | 0.158 | 9.5 |
| D1-helix2-helix3 | 0.19 | 0.138 | 1.4 |
| D3-helix1 | 0.1475 | 0.133 | 1.1 |
| D3-helix2 | 0.1695 | 0.128 | 1.3 |
| D3-helix3 | 0.155 | 0.137 | 1.1 |
| D3-helix1-helix2 | 0.274 | 0.245 | 1.1 |
| D3-helix2-helix3 | 0.247 | 0.213 | 1.2 |
| D3-helix3-loop1 | 0.229 | 0.135 | 1.7 |
| D3-loop1 | 0.1695 | 0.122 | 1.4 |
| D2-loop1 | 0.176 | 0.126 | 1.4 |
| D2-loop1-K | 0.195 | 0.158 | 1.2 |
| D2-loop3-K | 0.217 | 0.141 | 1.5 |
| D2-loop1-loop2 | 1.12 | 0.175 | 6.4 |
| D1-helix3 | 0.974 | 0.194 | 5.0 |
| D1-helix5 | 0.206 | 0.172 | 1.2 |
| D1-helix1-helix2 | 0.977 | 0.282 | 3.5 |
| D1-helix3-helix4 | 0.508 | 0.109 | 4.7 |
| D1-helix4-helix5 | 0.206 | 0.117 | 1.8 |
| D2-loop2-K | 0.160 | 0.124 | 1.3 |

Here, N represented the result of the positive control. A P/N ratio greater than 2 indicated antigen-antibody binding. The results demonstrated that the epitope peptides included D1-helix4, D2-loop1-loop2, D1-helix3, D1-helix1-helix2, and D1-helix3-helix4 peptides, with amino acid sequences as set forth in SEQ ID NO: 85, SEQ ID NO: 97, SEQ ID NO: 98, SEQ ID NO: 100, and SEQ ID NO: 101 respectively.

TABLE 14
Amino acid sequences of the epitope peptides

| No. | Peptide name | Amino acid sequence | SEQ ID No |
|---|---|---|---|
| 3 | D1-helix4 | SPKESPFRHVFWGSGSHTLPALLENLKLRKQNNGAF | 85 |
| 15 | D2-loop1-loop2 | GTKKDFEDLYTPVNGSIVIVRAGKITF | 97 |
| 16 | D1-helix3 | TDRFVMKKLNDRVMRVEYHFLSPYV | 98 |
| 18 | D1-helix1-helix2 | VRDLNQYRADIKEMGLSLOWLYSARGDFF | 100 |
| 19 | D1-helix3-helix4 | MRVEYHFLSPYVSPKESPFRHVFWGSGSHT | 101 |

The technical features of the above-described embodiments may be arbitrarily combined. To make the specification concise, not all possible combinations of the technical features in the above-described embodiments are described. However, they should be considered to be within the scope of this specification as long as there is no contradiction in the combination of these technical features.

The foregoing embodiments only show several implementations of the present disclosure and are described in detail, but they are not to be construed as a limit to the patent scope of the present disclosure. It should be noted that a person of ordinary skill in the art may further make variations and improvements without departing from the ideas of the present disclosure, and the variations and improvements shall fall within the protection scope of the present disclosure. Therefore, the patent protection scope of the present disclosure is subject to the protection scope of the appended claims.

What is claimed is:

1. An isolated human transferrin receptor (TfR) 1 antigen epitope peptide, wherein the isolated human TfR1 antigen epitope peptide has the amino acid sequence selected from: (1) SEQ ID NO: 85; (2) SEQ ID NO: 97; (3) SEQ ID NO: 98; (4) SEQ ID NO: 100; and (5) SEQ ID NO: 101.

2. A TfR1 antigen wherein the TfR1 antigen is a conjugate of the isolated human TfR1 antigen epitope peptide of claim 1 to a carrier protein.

3. A human TfR1 antibody wherein the human TfR1 antibody is an anti-TfR1 antibody produced using the TfR1 antigen of claim 2 as an immunogen.

4. An anti-TfR1 single-domain antibody, comprising a heavy chain variable region, wherein the heavy chain variable region comprises the heavy chain complementarity determining region (CDR) 1 as set forth in Jone of SEQ ID NO: 53 to SEQ ID NO: 60, the heavy chain CDR2 as set forth in one of SEQ ID NO: 61 to SEQ ID NO: 72, and the heavy chain CDR3 as set forth in one of SEQ ID NO: 73 to SEQ ID NO: 82.

5. The anti-TfR1 single-domain antibody of claim 4, wherein the heavy chain CDR1, the heavy chain CDR2, and the heavy chain CDR3 have the amino acid sequences selected from the group consisting of (1) to (12):
(1) the CDR1 as set forth in SEQ ID NO:53, the CDR2 as set forth in SEQ ID NO:62, and the CDR3 as set forth in SEQ ID NO:76;
(2) the CDR1 as set forth in SEQ ID NO:54, the CDR2 as set forth in SEQ ID NO:72, and the CDR3 as set forth in SEQ ID NO:80;
(3) the CDR1 as set forth in SEQ ID NO:55, the CDR2 as set forth in SEQ ID NO:64, and the CDR3 as set forth in SEQ ID NO:73;
(4) the CDR1 as set forth in SEQ ID NO:56, the CDR2 as set forth in SEQ ID NO:61, and the CDR3 as set forth in SEQ ID NO:75;
(5) the CDR1 as set forth in SEQ ID NO: 57, the CDR2 as set forth in SEQ ID NO: 66, and the CDR3 as set forth in SEQ ID NO: 74;
(6) the CDR1 as set forth in SEQ ID NO:58, the CDR2 as set forth in SEQ ID NO:68, and the CDR3 as set forth in SEQ ID NO:78;
(7) the CDR1 as set forth in SEQ ID NO:59, the CDR2 as set forth in SEQ ID NO:67, and the CDR3 as set forth in SEQ ID NO:82;
(8) the CDR1 as set forth in SEQ ID NO:59, the CDR2 as set forth in SEQ ID NO:63, and the CDR3 as set forth in SEQ ID NO:81;
(9) the CDR1 as set forth in SEQ ID NO:59, the CDR2 as set forth in SEQ ID NO:71, and the CDR3 as set forth in SEQ ID NO:77;
(10) the CDR1 as set forth in SEQ ID NO:59, the CDR2 as set forth in SEQ ID NO:69, and the CDR3 as set forth in SEQ ID NO:79;
(11) the CDR1 as set forth in SEQ ID NO:59, the CDR2 as set forth in SEQ ID NO:70, and the CDR3 as set forth in SEQ ID NO:77; and
(12) the CDR1 as set forth in SEQ ID NO:60, the CDR2 as set forth in SEQ ID NO:65, and the CDR3 as set forth in SEQ ID NO:73.

6. The anti-TfR1 single-domain antibody of claim 4, wherein the heavy chain variable region further comprises framework regions; the framework regions comprise a framework region (FR)1 region, an FR2 region, an FR3 region, and an FR4 region,
the FR1 region has the amino acid sequence as set forth in one of SEQ ID NOS: 25-33 or having at least about 60% sequence homology to one of SEQ ID NOS: 25-33;
the FR2 region has the amino acid sequence as set forth in one of SEQ ID NOS: 34-40 or having at least about 60% sequence homology to one of SEQ ID NOS: 34-40;
the FR3 region has the amino acid sequence as set forth in one of SEQ ID NOS: 41-51 or having at least about 60% sequence homology to one of SEQ ID NOS: 41-51; and
the FR4 region has the amino acid sequence as set forth in SEQ ID NO: 52 or having at least about 60% sequence homology to one of SEQ ID NO: 52, or
the anti-TfR1 single-domain antibody has the amino acid sequence as set forth in one of SEQ ID NOS: 1-12, or having at least about 80% sequence homology to one of SEQ ID NOS: 1-12.

7. (canceled)

8. The anti-TfR1 single-domain antibody of claim 4, wherein the anti-TfR1 single-domain antibody is a fragment crystallizable (Fc) fusion antibody, a humanized antibody, a chimeric antibody, a monoclonal antibody, or a polyclonal antibody.

9. A recombinant protein comprising an amino acid sequence of the anti-TfR1 single-domain antibody of claim 4.

10. The recombinant protein of claim 9, wherein the recombinant protein is a multi-epitope antibody, a multivalent antibody, or a multispecific antibody.

11. An antibody complex, comprising the anti-TfR1 single-domain antibody of claim 4 or a recombinant protein comprising an amino acid sequence of the anti-TfR1 single-domain antibody.

12. An antibody complex, wherein the antibody complex is a conjugate formed by conjugating the anti-TfR1 single-domain antibody of claim 4 or a recombinant protein comprising an amino acid sequence of the anti-TfR1 single-domain antibody to an active ingredient selected from a therapeutic agent, a diagnostic agent, a carrier protein, and a liposome.

13. A nucleic acid, encoding the anti-TfR1 single-domain antibody of claim 4 or a recombinant protein comprising an amino acid sequence of the anti-TfR1 single-domain antibody.

14. The nucleic acid of claim 13, wherein the nucleic acid has the nucleotide sequence as set forth in SEQ ID NOS: 13-24, or having at least about 80% sequence homology to one of SEQ ID NOS: 13-24.

15. An expression vector, comprising the nucleic acid of claim 13.

16. A host cell, comprising the expression vector of claim 15.

17. A pharmaceutical composition, comprising the anti-TfR1 single-domain antibody of claim 4, a recombinant protein comprising an amino acid sequence of the anti-TfR1 single-domain antibody, an antibody complex comprising the anti-TfR1 single-domain antibody or the recombinant protein, a nucleic acid encoding the anti-TfR1 single-domain antibody, an expression vector comprising the nucleic acid, or a host cell comprising the expression vector or the nucleic acid, and a pharmaceutically acceptable carrier.

18. (canceled)

19. (canceled)

20. A method for preventing or treating a disease associated with an abnormal TfR1 receptor expression, comprising administering to a subject in need thereof a therapeutically effective amount of the anti-TfR1 single-domain antibody of claim 4, a recombinant protein comprising an amino acid sequence of the anti-TfR1 single-domain antibody, an antibody complex comprising the anti-TfR1 single-domain antibody or the recombinant protein, a nucleic acid encoding the anti-TfR1 single-domain antibody, an expression vector comprising the nucleic acid, a host cell comprising the expression vector or the nucleic acid, or a pharmaceutical composition comprising at least one of the anti-TfR1 single-domain antibody, the recombinant protein, the antibody complex, the nucleic acid, the expression vector, the host cell, and a pharmaceutically acceptable carrier.

21. The method of claim 20, wherein the disease is a tumor abnormally expressing a TfR1 receptor.

22. The method of claim 21, wherein the tumor is a breast cancer, a glioma, a lung adenocarcinoma, a leukemia, lymphoma, a non-Hodgkin's lymphoma, or an ovarian cancer.

23. A kit for detecting a TfR1 receptor, comprising the anti-TfR1 single-domain antibody of claim 4, a recombinant protein comprising an amino acid sequence of the anti-TfR1 single-domain antibody, an antibody complex comprising the anti-TfR1 single-domain antibody or the recombinant protein, or a nucleic acid encoding the anti-TfR1 single-domain antibody.

24. (canceled)


SEQUENCE LISTING (103 sequences)

SEQ ID NO: 1 - protein, 109 aa, synthetic construct
ESGGGLVQPGESLRLSCVASGSIWSIYTMGWYRQAPGKQRDFVASITGDSTRTLESVKDRFIISRDNTKNTLYLQMNSLKPEDTAVYYCQAITSLGEYWGQGTQVTVSS

SEQ ID NO: 2 - protein, 110 aa, synthetic construct
ESGGGLVQPGESLRLSCAASGSIWSIYTMGWYRQAPGLQRDFVGSIAREGGRRYAESVKDRFTISRDNAKNTMYLQMNSLKREDTAVYYCNAITSLGEYWGQGTQVTVSS

SEQ ID NO: 3 - protein, 109 aa, synthetic construct
ESGGGLVQPGESLRLSCVASGSIWSIYTMGWYRQAPGKQRDFVASITRDSTRTLESVKDRFIISRDNAKNTLYLQMNSLKPEDTAVYYCHAITSLGEYWGQGTQVTVSS

SEQ ID NO: 4 - protein, 117 aa, synthetic construct
ESGGGLVQAGGSLTLSCAVSRRAFNTKAMGWFRQAPGKEREFVATISWSGFSTNYADSVKGRFTISRDNAKNTVYLQMNSLKPGDTAVYYCAAGVLDGTNWHEYDYWGQGTQVTVSS

SEQ ID NO: 5 - protein, 110 aa, synthetic construct
ESGGGLVQPGESLRLSCVASGSIWSIYTMGWYRQAPGKQRDFVASITRDGGRRHAESVKDRFTISRDNAKNTLYLQMNSLKPEDTAVYSCLAITSLGEYWGQGTQVTVSS

SEQ ID NO: 6 - protein, 113 aa, synthetic construct
ESGGGLVEPGGSLRLSCAASGNIFSINNMYWHRQAPGKQRELVGGITRGGITNYVDSVKGRFTISRDNAKNTVYLQMNSLKPEDTAVYFCNAHPWRLGIADYWGQGTQVTVSS

SEQ ID NO: 7 - protein, 110 aa, synthetic construct
ESGGGLVQPGESLRLSCVASGSIWSIYTMGWYRQAPGKQRDFVASITRDSGRRYAESVKDRFTISRDNAKNTLYLQTNSLKPEDTAVYYCHAITSLGEYWGQGTQVTVSS

SEQ ID NO: 8 - protein, 120 aa, synthetic construct
ESGGGLVQPGGSLRLSCATSGGTFEMYAMAWFRQAPGKDREGVAGFSWRDSSTTYYSDSVKGRFTISRDNAKNTVYLQMNSLKPEDTAVYYCAARPYVSRWSESSGYDYWGQGTQVTVSS

SEQ ID NO: 9 - protein, 118 aa, synthetic construct
ESGGGLVQAGASLKLSCNGSGRTFISYAMGWFRQAPGKQREFVAAERWSGSGIYYADSLKGRFTISRDNAKNTVYLEMNNLKPEDTAVYYCAAREGGLLHVATSYDYWGQGTQVTVSS

SEQ ID NO: 10 - protein, 111 aa, synthetic construct
ESGGGSVQPGGSLRLSCAVSGSIFSIDTMAWYRQAPGKQRELVAAITKGGSRNYADSVKGRFTISRDNAKNTVYLQMNSLKPEDTAVYYCLADRLFRDRSWGQGTQVTVSS

SEQ ID NO: 11 - protein, 117 aa, synthetic construct
ESGGGLVQPGGSLRLSCVASGRTFSTSGMGWFRQAPGKEREFVATISWSGFTTTYADSVKGRFTISRDNAKNTVYLQMNSLKPGDTAVYYCAAGVLDSTSWHEYDYWGQGTQVTVSS

SEQ ID NO: 12 - protein, 117 aa, synthetic construct
ESGGGLVQAGGSLRLSCAFSGPPFSTYGMGWFRQAPGKEREFVATISGSGFTTTYADSVKGRFTISRDNAKNTVYLQMNSLKPGDTAVYYCAAGVLDGTNWHEYDYWGQGTQVTVSS

SEQ ID NO: 13 - DNA, 327 bp, synthetic construct
  source 1..327: mol_type = other DNA
gagtctgggggaggcttggtgcagcctggggagtctctgagactctcctgtgtagcctctggaagcatctggagtatctataccatgggctggtaccgccaggctccagggaagcagcgcgacttcgtcgcaagtattacaggtgatagtactaggacgttagagtccgtgaaggaccgatttattatctccagagacaacaccaagaacacgttgtatctgcaaatgaacagtctgaaacctgaggacacggccgtctattactgtcaggcaatcacttcgcttggagaatactggggccaggggacccaggtcaccgtctcctca

SEQ ID NO: 14 - DNA, 330 bp, synthetic construct
  source 1..330: mol_type = other DNA
gagtctgggggaggcttggtgcagcctggggagtctctgagactctcctgtgcagcctctggaagcatctggagtatctataccatgggctggtaccgccaggctccaggactgcagcgcgacttcgtcggaagtatagcgcgtgaaggtggtaggagatatgcagagtccgtgaaggaccgattcaccatctccagagacaacgccaagaacacgatgtatctgcaaatgaacagtctgaaacgtgaagacacggccgtctattactgtaatgcaatcacttcgcttggagaatactggggccaggggacccaggtcaccgtctcctca

SEQ ID NO: 15 - DNA, 327 bp, synthetic construct
  source 1..327: mol_type = other DNA
gagtctggaggaggcttggtgcagcctggggagtctctgagactctcctgtgtagcctctggaagcatctggagtatctataccatgggctggtaccgccaggctccagggaagcagcgcgacttcgtcgcaagtattacgcgtgatagtactaggacgttagagtccgtgaaggaccgatttattatctccagagacaacgccaagaacacgttgtatctgcaaatgaacagtctgaaacctgaggacacggccgtctattactgtcatgcaatcacttcgcttggagaatactggggccaggggacccaggtcaccgtctcctca

SEQ ID NO: 16 - DNA, 351 bp, synthetic construct
  source 1..351: mol_type = other DNA
gagtctgggggaggattggtgcaggctgggggctctctgacactctcctgtgcagtctctcgacgcgccttcaataccaaagctatgggctggttccgccaggctccagggaaggagcgtgaatttgtagcaactattagttggagtggttttagcacaaactatgcagactccgtgaagggccgattcaccatctccagagacaacgccaagaacacggtgtatctgcaaatgaacagcctgaaacctggggacacggccgtttattactgtgcagcgggggtcttagacggtactaattggcatgaatatgactactggggccaggggacccaggtcaccgtctcctca

SEQ ID NO: 17 - DNA, 330 bp, synthetic construct
  source 1..330: mol_type = other DNA
gagtctggaggaggcttggtgcagcctggggagtctctgagactctcctgtgtagcctctggaagcatctggagtatctataccatgggctggtaccgccaggctccagggaagcagcgcgacttcgtcgcaagtattacgcgtgatggtggtaggcgccatgcagagtccgtgaaggaccgattcaccatctccagagacaacgccaagaacacgttgtatctgcaaatgaacagtctgaaacctgaggacacggccgtctattcgtgccttgcaatcacttcgcttggagaatactggggccaggggacccaggtcaccgtctcctca

SEQ ID NO: 18 - DNA, 339 bp, synthetic construct
  source 1..339: mol_type = other DNA
gagtctgggggaggcttggtggagcctggggggtctctgagactctcctgtgcagcctctggaaacatcttcagtatcaataacatgtactggcaccgccaggctccagggaagcagcgcgagctggtcggaggtattactagaggtggtatcacaaactatgtagactccgtgaagggccgattcaccatctccagagacaacgccaagaacacggtgtatctgcaaatgaacagcctgaaacctgaggacacggccgtctatttctgtaatgcccacccgtggcggcttgggattgctgactactggggccaggggacccaggtcaccgtctcctca

SEQ ID NO: 19 - DNA, 330 bp, synthetic construct
  source 1..330: mol_type = other DNA
gagtctggaggaggcttggtgcagcctggggagtctctgagactctcctgtgtagcctctggaagcatctggagtatctataccatgggctggtaccgccaggctccagggaagcagcgcgacttcgtcgcaagtattacgcgtgatagtggtaggaggtatgcagagtccgtgaaggaccgattcaccatctccagagacaacgccaagaacacgttgtatctgcaaacgaacagtctgaaacctgaggacacggccgtctattactgtcatgcaatcacttcgcttggagaatactggggccaggggacccaggtcaccgtctcctca

SEQ ID NO: 20 - DNA, 360 bp, synthetic construct
  source 1..360: mol_type = other DNA
gagtctggaggaggcttggtgcagcctggggggtctctgagactctcctgtgcaacctctggtggcaccttcgaaatgtatgccatggcgtggttccgccaggctccagggaaggatcgtgagggtgtagcaggttttagctggagagatagtagtacaacatactattcagactccgtgaagggccgattcaccatctccagagacaacgccaagaacacggtgtatctgcaaatgaacagcctgaaacctgaagacacggccgtttattactgtgcagcccgaccgtacgttagtaggtggtcggaatcaagcgggtatgactactggggccaggggacccaggtcaccgtctcctca

SEQ ID NO: 21 - DNA, 354 bp, synthetic construct
  source 1..354: mol_type = other DNA
gagtctggaggaggattggtgcaggctggggcctctctgaaactctcctgtaatggttctggacgcaccttcattagctatgccatgggctggttccgccaggctccagggaagcagcgtgagtttgttgcagctgagaggtggagtggtagtggcatatactatgcagactccttgaagggccgattcaccatctccagagacaacgccaagaacacggtgtatctggaaatgaacaacctgaaacctgaggacacggccgtttattactgtgcagcacgtgagggaggattactgcatgtagcgacttcgtatgactactggggccaggggacccaggtcaccgtctcctca

SEQ ID NO: 22 - DNA, 333 bp, synthetic construct
  source 1..333: mol_type = other DNA
gagtctggaggaggctcggtgcagcctggggggtctctgagactctcctgtgcagtctctggaagcatcttcagtatcgataccatggcctggtaccgccaggctccagggaagcagcgcgagttggtcgcagctattactaagggtggtagtagaaactatgcagactccgtgaagggccgattcaccatctccagagacaacgccaagaacacggtgtatctacaaatgaacagcctgaaacctgaggacacagccgtctattactgtctagcagatagactcttccgagatcgttcctggggccaggggacccaggtcaccgtctcctca

SEQ ID NO: 23 - DNA, 351 bp, synthetic construct
  source 1..351: mol_type = other DNA
gagtctggaggaggattggtgcagcctggggggtctctgagactttcctgtgtagcctctggacgcaccttcagtacctccggcatgggctggttccgccaggctccagggaaggagcgtgaatttgtagcaactattagttggagtggttttacgacaacctatgcagactccgtgaagggccgattcaccatctccagagacaacgccaagaacacggtgtatctgcaaatgaacagcctgaaacctggggacacggccgtttattactgtgcagcgggggtcttagacagtactagttggcatgaatatgactactggggccaggggacccaggtcaccgtctcctca

SEQ ID NO: 24 - DNA, 351 bp, synthetic construct
  source 1..351: mol_type = other DNA
gagtctggaggaggattggtgcaggctgggggctctctgagactctcctgtgcattctctggaccccccttcagtacctatggcatgggctggttccgccaggctccagggaaggagcgtgaatttgtagcaactattagtgggagtggttttacaacaacctatgcagactccgtgaagggccgattcaccatctccagagacaacgccaagaacacggtgtatctgcaaatgaacagcctgaaacctggggacacggccgtttattactgtgcagcgggggtcttagatggtactaattggcatgaatatgactactggggccaggggacccaggtcaccgtctcctca

SEQ ID NO: 25 - protein, 20 aa, synthetic construct
ESGGGLVEPGGSLRLSCAAS

SEQ ID NO: 26 - protein, 20 aa, synthetic construct
ESGGGLVQAGASLKLSCNGS

SEQ ID NO: 27 - protein, 20 aa, synthetic construct
ESGGGLVQAGGSLRLSCAFS

SEQ ID NO: 28 - protein, 20 aa, synthetic construct
ESGGGLVQAGGSLTLSCAVS

SEQ ID NO: 29 - protein, 20 aa, synthetic construct
ESGGGLVQPGESLRLSCAAS

SEQ ID NO: 30 - protein, 20 aa, synthetic construct
ESGGGLVQPGESLRLSCVAS

SEQ ID NO: 31 - protein, 20 aa, synthetic construct
ESGGGLVQPGGSLRLSCATS

SEQ ID NO: 32 - protein, 20 aa, synthetic construct
ESGGGLVQPGGSLRLSCVAS

SEQ ID NO: 33 - protein, 20 aa, synthetic construct
ESGGGSVQPGGSLRLSCAVS

SEQ ID NO: 34 - protein, 17 aa, synthetic construct
MAWFRQAPGKDREGVAG

SEQ ID NO: 35 - protein, 17 aa, synthetic construct
MAWYRQAPGKQRELVAA

SEQ ID NO: 36 - protein, 17 aa, synthetic construct
MGWFRQAPGKEREFVAT

SEQ ID NO: 37 - protein, 17 aa, synthetic construct
MGWFRQAPGKQREFVAA

SEQ ID NO: 38 - protein, 17 aa, synthetic construct
MGWYRQAPGKQRDFVAS

SEQ ID NO: 39 - protein, 17 aa, synthetic construct
MGWYRQAPGLQRDFVGS

SEQ ID NO: 40 - protein, 17 aa, synthetic construct
MYWHRQAPGKQRELVGG

SEQ ID NO: 41 - protein, 38 aa, synthetic construct
NYADSVKGRFTISRDNAKNTVYLQMNSLKPEDTAVYYC

SEQ ID NO: 42 - protein, 38 aa, synthetic construct
NYADSVKGRFTISRDNAKNTVYLQMNSLKPGDTAVYYC

SEQ ID NO: 43 - protein, 38 aa, synthetic construct
NYVDSVKGRFTISRDNAKNTVYLQMNSLKPEDTAVYFC

SEQ ID NO: 44 - protein, 38 aa, synthetic construct
RHAESVKDRFTISRDNAKNTLYLQMNSLKPEDTAVYSC

SEQ ID NO: 45 - protein, 38 aa, synthetic construct
RTLESVKDRFIISRDNAKNTLYLQMNSLKPEDTAVYYC

SEQ ID NO: 46 - protein, 38 aa, synthetic construct
RTLESVKDRFIISRDNTKNTLYLQMNSLKPEDTAVYYC

SEQ ID NO: 47 - protein, 38 aa, synthetic construct
RYAESVKDRFTISRDNAKNTLYLQTNSLKPEDTAVYYC

SEQ ID NO: 48 - protein, 38 aa, synthetic construct
RYAESVKDRFTISRDNAKNTMYLQMNSLKREDTAVYYC

SEQ ID NO: 49 - protein, 38 aa, synthetic construct
TYADSVKGRFTISRDNAKNTVYLQMNSLKPGDTAVYYC

SEQ ID NO: 50 - protein, 38 aa, synthetic construct
YYADSLKGRFTISRDNAKNTVYLEMNNLKPEDTAVYYC

SEQ ID NO: 51 - protein, 38 aa, synthetic construct
YYSDSVKGRFTISRDNAKNTVYLQMNSLKPEDTAVYYC

SEQ ID NO: 52 - protein, 11 aa, synthetic construct
WGQGTQVTVSS

SEQ ID NO: 53 - protein, 8 aa, synthetic construct
GGTFEMYA

SEQ ID NO: 54 - protein, 8 aa, synthetic construct
GNIFSINN

SEQ ID NO: 55 - protein, 8 aa, synthetic construct
GPPFSTYG

SEQ ID NO: 56 - protein, 8 aa, synthetic construct
GRTFISYA

SEQ ID NO: 57 - protein, 8 aa, synthetic construct
GRTFSTSG

SEQ ID NO: 58 - protein, 8 aa, synthetic construct
GSIFSIDT

SEQ ID NO: 59 - protein, 8 aa, synthetic construct
GSIWSIYT

SEQ ID NO: 60 - protein, 8 aa, synthetic construct
RRAFNTKA

SEQ ID NO: 61 - protein, 8 aa, synthetic construct
ERWSGSGI

SEQ ID NO: 62 - protein, 9 aa, synthetic construct
FSWRDSSTT

SEQ ID NO: 63 - protein, 7 aa, synthetic construct
IAREGGR

SEQ ID NO: 64 - protein, 8 aa, synthetic construct
ISGSGFTT

SEQ ID NO: 65 - protein, 8 aa, synthetic construct
ISWSGFST

SEQ ID NO: 66 - protein, 8 aa, synthetic construct
ISWSGFTT

SEQ ID NO: 67 - protein, 6 aa, synthetic construct
ITGDST

SEQ ID NO: 68 - protein, 7 aa, synthetic construct
ITKGGSR

SEQ ID NO: 69 - protein, 7 aa, synthetic construct
ITRDGGR

SEQ ID NO: 70 - protein, 7 aa, synthetic construct
ITRDSGR

SEQ ID NO: 71 - protein, 6 aa, synthetic construct
ITRDST

SEQ ID NO: 72 - protein, 7 aa, synthetic construct
ITRGGIT

SEQ ID NO: 73 - protein, 15 aa, synthetic construct
AAGVLDGTNWHEYDY

SEQ ID NO: 74 - protein, 15 aa, synthetic construct
AAGVLDSTSWHEYDY

SEQ ID NO: 75 - protein, 16 aa, synthetic construct
AAREGGLLHVATSYDY

SEQ ID NO: 76 - protein, 17 aa, synthetic construct
AARPYVSRWSESSGYDY

SEQ ID NO: 77 - protein, 9 aa, synthetic construct
HAITSLGEY

SEQ ID NO: 78 - protein, 10 aa, synthetic construct
LADRLFRDRS

SEQ ID NO: 79 - protein, 9 aa, synthetic construct
LAITSLGEY

SEQ ID NO: 80 - protein, 12 aa, synthetic construct
NAHPWRLGIADY

SEQ ID NO: 81 - protein, 9 aa, synthetic construct
NAITSLGEY

SEQ ID NO: 82 - protein, 9 aa, synthetic construct
QAITSLGEY

SEQ ID NO: 83 - protein, 28 aa, synthetic construct
NLDYERYNSQLLSFVRDLNQYRADIKEM

SEQ ID NO: 84 - protein, 30 aa, synthetic construct
GLSLQWLYSARGDFFRATSRLTTDFGNAEK

SEQ ID NO: 85 - protein, 36 aa, synthetic construct
SPKESPFRHVFWGSGSHTLPALLENLKLRKQNNGAF

SEQ ID NO: 86 - protein, 28 aa, synthetic construct
RATSRLTTDFGNAEKTDRFVMKKLNDRV

SEQ ID NO: 87 - protein, 19 aa, synthetic construct
RLYWDDLKRKLSEKLDSTD

SEQ ID NO: 88 - protein, 14 aa, synthetic construct
FTSTIKLLNENSYV

SEQ ID NO: 89 - protein, 22 aa, synthetic construct
EAGSQKDENLALYVENQFREFK

SEQ ID NO: 90 - protein, 33 aa, synthetic construct
RLYWDDLKRKLSEKLDSTDFTSTIKLLNENSYV

SEQ ID NO: 91 - protein, 38 aa, synthetic construct
FTSTIKLLNENSYVPREAGSQKDENLALYVENQFREFK

SEQ ID NO: 92 - protein, 33 aa, synthetic construct
EAGSQKDENLALYVENQFREFKLSKVWRDQHFV

SEQ ID NO: 93 - protein, 11 aa, synthetic construct
LSKVWRDQHFV

SEQ ID NO: 94 - protein, 10 aa, synthetic construct
GTKKDFEDLY

SEQ ID NO: 95 - protein, 20 aa, synthetic construct
VHANFGTKKDFEDLYTPVNG

SEQ ID NO: 96 - protein, 20 aa, synthetic construct
NMEGDCPSDWKTDSTCRMVT

SEQ ID NO: 97 - protein, 27 aa, synthetic construct
GTKKDFEDLYTPVNGSIVIVRAGKITF

SEQ ID NO: 98 - protein, 25 aa, synthetic construct
TDRFVMKKLNDRVMRVEYHFLSPYV

SEQ ID NO: 99 - protein, 32 aa, synthetic construct
NETLFRNQLALATWTIQGAANALSGDVWDIDN

SEQ ID NO: 100 - protein, 29 aa, synthetic construct
VRDLNQYRADIKEMGLSLQWLYSARGDFF

SEQ ID NO: 101 - protein, 30 aa, synthetic construct
MRVEYHFLSPYVSPKESPFRHVFWGSGSHT

SEQ ID NO: 102 - protein, 34 aa, synthetic construct
LPALLENLKLRKQNNGAFNETLFRNQLALATWTI

SEQ ID NO: 103 - protein, 20 aa, synthetic construct
SIVIVRAGKITFAEKVANAE